(19) United States
(12) Patent Application Publication
(10) Pub. No.: US 2023/0295088 A1
(43) Pub. Date: Sep. 21, 2023

(54) INSULIN SENSITIZERS FOR THE TREATMENT OF DIABETES MELLITUS

(71) Applicant: University of Iowa Research Foundation, lowa City, IA (US)

(72) Inventors: Julien A. Sebag, lowa City, IA (US); Terry C. Yin, Cedar Rapids, IA (US); Robert J. Kerns, Iowa City, IA (US)

(21) Appl. No.: 18/017,826

(22) PCT Filed: Aug. 12, 2021
(86) PCT No.: PCT/US2021/045757

§ 371 Date: Jan. 24, 2023

Related U.S. Application Data
(60) Provisional application No. 63/064,737, filed on Aug. 12, 2020.

(51) Int. Cl. C07D 211/04; G01N 33/50; A01K 67/027; C07C 211/45; A61P 3/10

(52) U.S. Cl. CPC C07D 211/04; G01N 33/5008; A01K 67/0275; C07C 211/45; A61P 3/10; G01N 2333/62; A01K 2227/10; A01K 2267/0362

(57) ABSTRACT

Compositions and methods for insulin sensitization are provided.

### CROSS-REFERENCE TO RELATED APPLICATIONS

This application claims the benefit of the filing date of U.S. application No. 63/064,737, filed on Aug. 12, 2020, the disclosure of which is incorporated by reference herein.

### BACKGROUND

Insulin promotes the storage of glucose in skeletal muscle and adipose tissue by triggering the translocation of GLUT4 glucose transporters from intracellular storage vesicles to the plasma membrane of myocytes and adipocytes. The decrease in insulin's ability to initiate this process is referred to as insulin resistance and eventually leads to the development of type 11 diabetes mellitus (T2D). Potentiating the effect of insulin on GLUT4 translocation would be greatly beneficial for correcting insulin resistance and to prevent or treat T2D,

### SUMMARY

Molecules were identified that enhanced insulin-mediated GLUT4 translocation, which can improve glycemic control and/or treat type II diabetes. Specifically, a luciferase recombination technology, NanoBit, was used to generate a homogenous GLUT4 translocation assay for high throughput screening (HTS). Using this assay, a library of about 50,000 small molecules was screened to identify compounds that enhance insulin-stimulated GLUT4 translocation, leading to a focus on 80 of the molecules. A mouse model was engineered with the same technology to validate the compounds identified on primary adipocytes and primary muscle fibers, in live animals.

For example, some of the compounds, C3 and derivatives thereof, e.g., C59, enhanced the effect of insulin on GLUT4 translocation and glucose uptake. In contrast to current treatments for diabetes, which largely fail to achieve glycemic control and whose mode of action is poorly understood, the disclosed compounds require insulin, e.g., endogenous insulin, and enhance its actions. For that reason, those compounds are not likely to cause hypoglycemia during fasting due to the low levels of circulating insulin and can potentiate the glucose clearing effect of insulin postprandially when circulating insulin is high. This greatly improves glycemic control in diabetic patients. Also, unlike thiazolidinediones (TZDs), which cause severe side effects due to their agonist activity on PPARg, the disclosed compounds have little if any activity at PPARg, and unlike metformin, they are actual insulin sensitizers. Thus, the compounds described herein are useful for the treatment of type II diabetes in humans, e.g., as they potentiate the effect of insulin on GLUT4 translocation, and are active, e.g., in vivo, in the presence of endogenous insulin and consequently enhance its action while maintaining the physiological spacio-temporal action of insulin.

In one embodiment, a method for preventing, inhibiting or treating type II diabetes in a mammal is provided. In one embodiment, the method employs a compound that targets UNC119 and/or UNC119b. In one embodiment, the method employs a compound of one of formulas (I)-(III). In one embodiment, the mammal is a human.

In one embodiment, a method providing for or enhancing glycernic control in a mammal is provided. In one embodiment, the method employs a compound that targets UNC119 and/or UNC119b. In one embodiment, the method employs a compound of one of formulas (I)-(III). In one embodiment, the mammal is a human.

In one embodiment, a method for enhancing insulin-stimulated GLUT4 translocation in a mammal is provided. In one embodiment, the method employs a compound that targets UNC119 and/or UNC119b. In one embodiment, the method employs a compound of one of formulas (I)-(III). In one embodiment, the mammal is a human.

In one embodiment, a method for preventing, inhibiting or treating insulin resistance in a mammal is provided. In one embodiment, the method employs a compound that targets UNC119 and/or UNC119b. In one embodiment, the method employs a compound of one of formulas (I)-(III). In one embodiment, the mammal is a human.

In one embodiment, a method for sensitizing a mammal to insulin levels is provided. In one embodiment, the method employs a compound that targets UNC119 and/or UNC119b. In one embodiment, the method employs a compound of one of formulas (I)-(III). In one embodiment, the mammal is a human.

In one embodiment, the method includes administering to a mammal in need thereof an effective amount of a compound of formula (I) having the structure:

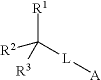

wherein

each of R1, R2, and R3 is independently H, alkyl, aryl, or heteroaryl, or two or three of R1, R2, and R3 taken together with the carbon to which they, attach form a cycloalkyl ring;

L is a linker selected from amide, oxime, carbamate, carbamide, triazole, oxazole, oxadiazole, or sulfonamide; and

A is aryl or heteroaryl.

In one embodiment, A is substituted aryl, substituted heteroaryl, or unsubstituted heteroaryl. In one embodiment, A is substituted with one, two, or three chloro. In one embodiment, A is a heteroaryl. In one embodiment, A is pyridine, pyrimidine, pyridazine, pyrazine, quinoline, isoquinoline, or naphthylene. In one embodiment, A is a dichlorophenyl, optionally substituted with one or more additional substituent. In one embodiment A is other than phenyl. In one embodiment, A is 2-pyridinyl, 2-pyrimidinyl, or 3-isoquinolyl. In one embodiment, A is a 6-membered ring has a halogen para to the linkage to L. In one embodiment, A is a 6-membered ring having a 4-halo, 5-halo, or both. In one embodiment, A is a 6-membered ring having a 4-chloro, 5-chloro, or both, In one embodiment, A is a 6-membered heteroaryl having a 4-chloro, 5-chloro, or both.

In one embodiment, the method includes administering to a mammal in need thereof an effective amount of a compound of formula (II) having the structure:

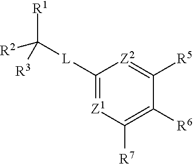

wherein

each of R¹, R², and R³ is independently alkyl, aryl, or heteroaryl, or two or three of R1, R², and R³ taken together with the carbon to which they attach form a cycloalkyl ring;

L is a linker selected from amide; oxime, carbamate, sulfonamide, or carbamide;

each of Z¹ and Z² is independently CR⁸, CR⁹, or N;

each of R⁵, R⁶, R⁷, R⁸, and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarhonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring; and

when Z¹ and Z² are other than N, then at least one of R⁵, R⁶, R⁷, R⁸, and R⁹ is other than hydrogen.

In one embodiment, the method includes administering to a mammal in need thereof an effective amount of a compound of formula (III) having the structure:

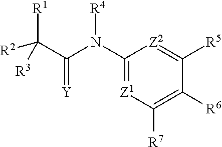

wherein

each of R¹, R², and R³ is independently H, alkyl, aryl, or heteroaryl, or two or three of R1, R², and R³ taken together with the carbon to which they attach form a cycloalkyl ring;

L is a linker selected from amide, oxime, carbamate, sulfonamide, or carbamide;

Y is O, S, or NR10,

each of R4 and R10 is independently hydrogen, alkyl, aryl, hydroxyl;

each of Z¹ and Z² is independently CR⁸, CR^{9,} or N;

each of R⁵, R⁶, R⁷, R⁸, and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring; and

when Z¹ and Z² are other than N, then at least one two of R⁵, R⁶, R⁷, R⁸, and R⁹ is other than hydrogen.

Also provided is a compound of formula (II) having the structure:

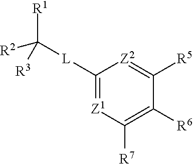

wherein

each of R¹, R², and R³ is independently H, alkyl, aryl, or heteroaryl, or two or three of R¹, R², and R³ taken together with the carbon to which they attach form a cycloalkyl ring;

L is a linker selected from amide, oxime, carbamate, carbamide, triazole, oxazole, oxadiazole, or sulfonamide;

each of Z¹ and Z² is independently CR⁸, CR⁹, or N;

each of R⁵, R⁷, R⁸, and R^{9,} if present, is independently H, fluoro, chloro, bromo, iodo, amino, amino, alkyl, alkoxy, alkylamino, alkylthio, alkylamido, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring; and
when Z¹ and Z² are CR⁵ or CR⁹, or when R¹, R², and R3 taken together are adamantyl, then at least one two of R⁵, R⁶, R⁷, R⁸, and R⁹ is other than hydrogen, and at least one of R⁵, R⁶, R⁷, R⁸, and R⁹ other than fluoro, chloro, bromo, and iodo. In one embodiment, a compound of formula (II) is not C3.

Further provided is a compound of formula (III) having the structure:

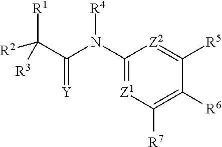

wherein

each of R¹, R², and R3 is independently H, alkyl, aryl, or heteroaryl, or two or three of R1, R², and R³ taken together with the carbon to which they attach form a cycloalkyl ring;

L is a linker selected from amide, oxime, carbamate, sulfonamide, or carbamide;

Y is O, S, or NR10;

each of R4 and R10 is independently hydrogen, alkyl, aryl, hydroxyl;

each of Z¹ and Z² is independently CR⁸, CR⁹, or N;

each of R⁵, R⁶, R⁷, R⁸, and R⁹, if present, is independently fluoro, chloro, bromo, iodo, amino, amino, alkyl, alkoxy, alkylamino, alkylthio, alkylamido formyl, acyl, alkoxycarbonyl, acyloxy, aryloxy, arylamino, arylthio, arylcarbonyl, arylamido; aryloxycarbonyl, hydroxy, amino, cyano, nitro, azide, thio, alkylsulfonyl, alkylsulfinyl, carbonate, sulfonate, or phosphonate, or two of is R⁵, R⁶, R⁷, R⁸, and R⁹ taken together with the carbons to which they attach form an aryl or heteroaryl ring; and

when Z¹ and Z² are CR⁸ or CR⁹, or when R¹, R², and R3 taken together are adamantyl, then at least one two of R⁵, R⁶, R⁷, R⁸, and R⁹ is other than hydrogen, and at least one of R⁵, R⁶, R⁷, R⁸, and R⁹ other than fluoro, chloro, bromo, and iodo. In one embodiment, a compound of formula (III) is not C3.

In one embodiment, Y is O, or —N—OH. In one embodiment, three of R¹, R², and R3 taken together with the carbon to which they attach form a cycloalkyl. In one embodiment, R¹, R², and R3 taken together with the carbon to which they attach form an adamantyl ring. In one embodiment, two of R¹, R², and R3 taken together with the carbon to which they attach form a cycloalkyl, one embodiment, two of R¹, R², and R3 taken together with the carbon to which they attach form a cyclohexane or tetrahydropyran. In one embodiment, at least one of Z¹ and Z² is N. In one embodiment, Z¹ and Z² are independently CR⁸ or CR⁹ and at least two of R⁵, R⁶, R⁷, R⁸, and R⁹ are chloro and at least one of R⁵, R⁶, R⁷, R⁸, and R⁹ are other than chloro and other than hydrogen. In one embodiment, at least two of R⁵, R⁶, R⁷, R⁸, and R⁹ is chloro, fluoro, bromo, or iodo. In one embodiment; R⁶ is chloro, fluoro, bromo, or iodo. In one embodiment, one of R⁵ and R⁷ is methyl, chloro, flonoro, or bromo. In one embodiment, the compound has the structure:

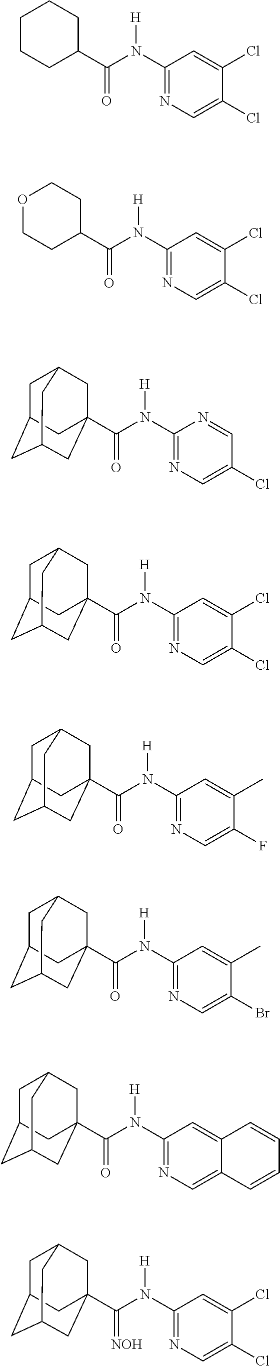

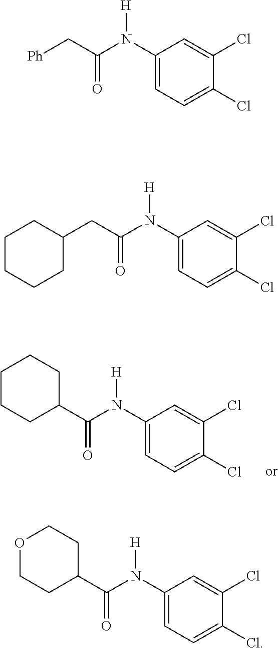 [SMILES: O=C(Cc1ccccc1)Nc1ccc(Cl)c(Cl)c1 ; O=C(CC1CCCCC1)Nc1ccc(Cl)c(Cl)c1 ; O=C(Nc1ccc(Cl)c(Cl)c1)C1CCCCC1 ; O=C(Nc1ccc(Cl)c(Cl)c1)C1CCOCC1]

In one embodiment, the compound is

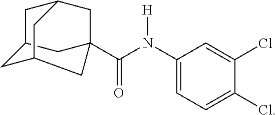 [SMILES: O=C(Nc1ccc(Cl)c(Cl)c1)C12CC3CC(CC(C3)C1)C2]

### BRIEF DESCRIPTION OF THE FIGURES

FIGS. 1A-1M. HTS identification of insulin sensitizers, A) Schematic depicting HiBiT-GLUT4-mCherry. B) GLUT4 localization in HBG4 cells using mCherry fluorescence microscopy. C) HiBiT luciferase complementation assay used for measuring GLUT4 surface density. D) Real-time GLUT4 translocation in response to increasing concentrations of insulin (0 to 100 nM) using the HiBiT assay. E) Insulin concentration-response of GLUT4 translocation in HBG4 cells. F) Results from primary screen and validation. G) C3 formula. H) Surface GLUT4 density in HBG4 cells treated with vehicle or 100 nM insulin and increasing concentrations of C3. I) Insulin concentration-response of GLUT4 translocation in HBG4 cells in the presence or absence of C3. J) GTT in lean mice 10 minutes after injection of vehicle or C3. (n=20 per group) K) GTT in DIO mice treated with vehicle or C3 for two weeks (n=15 per group). L) fasting blood glucose in DIO mice after 2 weeks treatment with vehicle or C3. (n=5) M) Fasting plasma insulin in DIO mice after 2 weeks treatment with vehicle or C3. (n=5). *** p<0.001.

FIGS. 2A-2P. SAR and in-vivo efficacy of C3 derivatives. A-D) Insulin-stimulated. GLUT4 translocation in the presence of vehicle or derivatives of C3. E) structure of C59 (C3 derivative), F-G) GLUT4 translocation in response to increasing concentration of insulin in the absence (F) or presence (G) of 10 μM C59. I) Insulin concentration response curve in the presence of vehicle or C59. I) Insulin concentration response curve in the presence of vehicle, C3 or C59. J) GLUT4 translocation in response to increasing concentration of C59 in the absence or presence of 100 nM insulin. K) GTT in lean mice treated with vehicle or C59 10 minutes before glucose injection. (n=15-18 per group). L) GTT in DIO mice treated IP with vehicle or C59. (n=11 -13 per group). M) GTT in DIO mice treated orally with vehicle or C59. (n=10 per group). N) Fasting blood glucose in DIO mice after 2 weeks oral treatment with vehicle or C59. (n=10 per group). O) Fasting plasma insulin in DIO mice after 2 weeks oral treatment with vehicle or C59. (n=10 per group). P) PPARγ activity in cells treated with vehicle, rosiglitazone, C3 or C59. ***p<0.001.

FIGS. 3A-3H. Mechanism of C59 insulin sensitization in vivo. A-C) Insulin-stimulated GLUT4 translocation measurement in HBG4 DIO mice treated with vehicle or C59, luminescence was acquired using an IVIS system and quantifications are shown in B and C. (n=6-10 per group). D-H) PET/CT scan measurement of F-FDG in DIO mice treated with vehicle or C59. (n=5 per group). PET/CT images are shown in (D) followed by quantitation of F-FDG uptake in fore limbs (E), hind limbs (F), brown adipose tissue (G) and heart (H). *p<0.05, **p<0.01, ***p<0.001.

FIGS. 4A-4K. Identification and validation of C59 target. A) Cells-based PISA results showing concentration dependent increased thermostability of Unc119 and Unc119b in the presence of C59. B) Cells-based and crude extract PISA showing increased thermostability of Unc119 and Unc119b in the presence of C59 and C23 compared to controls DMSO and C29. C-D) Luminescence complementation based thermal shift assay showing increased thermostabillity of Unc119 (C) and UNC119b (D) by C59. E) PCR genotyping of Unc119 or Unc119b deletion in Unc119 and Unc119b KO HBG4 cell lines generated using CRISPR CasP. F-H) Insulin stimulated GLUT4 translocation in the presence (red) or absence (black) of C59 in WT (F). Unc119 KO (G) and UNC119b KO (H) HBG4 cells I-K. H-Deoxy-D-Glucose uptake in WT (I), Unc119 KO (J) and Unc119b KO(K) HBG4 cells treated with vehicle; insulin, C59 and insulin supplemented with. C59. *p<0.05, **p<0.01, ***p<0.001, ****p<0.0001.

### DETAILED DESCRIPTION

### Definitions

The term “alkyl” as used herein refers to substituted or unsubstituted straightchain, branched or cyclic, saturated mono- or bivalent groups having from 1 to 20 carbon atoms, 10 to 20 carbon atoms, 12 to 18 carbon atoms, 6 to about 10 carbon atoms, 1 to 10 carbons atoms, 1 to 8 carbon atoms. 2 to 8 carbon atoms, 3 to 8 carbon atoms, 4 to 8 carbon atoms, 5 to 8 carbon atoms, 1 to 6 carbon atoms. 2 to 6 carbon atoms, 3 to 6 carbon atoms, or 1 to 3 carbon atoms. Examples of straight chain mono-valent (C₁-C₂₀)-alkyl groups include those with from 1 to 8 carbon atoms such as methyl (i.e., CH₃), ethyl, n-propyl, n-butyl, n-pentyl, n-hexyl, n-heptyl, n-octyl groups. Examples of branched mono-valent (C₁-C₂₀)-alkyl groups include isopropyl, iso-butyl, sec-butyl, t-butyl, neopentyl, and isopentyl. Examples of straight chain bi-valent (C₁-C₂₀)alkyl groups include those with from 1 to 6 carbon atoms such as —CH₂—, —CH₂CH₂—, —CH₂CH₂CH₂—, —CH₂CH₂CH₂CH₂-, and —CH₂CH₂CH₂CH₂CH₂-. Examples of branched bi-valent alkyl groups include —CH(CH₃)CH₂- and —CH₂CH(CH₃)CH₂-. Examples of cyclic alkyl groups include cyclopropyl, cyclobutyl, cyclopently, cyclohexyl, cyclooctyl; bicyclo[1.1.1]pentyl, bicyclo[2.1.1]hexyl, bicyclo[2.2.1]heptyl, and adamantyl. Cycloalkyl groups further include substituted and unsubstituted polycyclic cycloalkyl groups such as, but not limited to, norbornyl, adamantyl, bornyl, camphenyl, isocamphenyl, and carenyl groups, and fused rings such as, but not limited to, decalinyl, and the like. For example cycloalkyl includes an adamantyl substituted by one, two, three, four, or more substituents, e.g., at the tertiary bridgehead positions at the methylene bridges. In various examples, cycloalkyl can be substituted with one or more heteroatoms to provide a heterocycloalkyl, e.g., a morpholine, tetrahydropyran, or pyrrolidine ring. Alkyl includes alkyenyl, alkynyl and other derivatives of alkyl having one or more double bond or triple bond.

The term “aryl” as used herein refers to substituted or unsubstituted univalent groups that are derived by removing a hydrogen atom from an areae, which is a cyclic aromatic hydrocarbon, having from 6 to 20 carbon atoms, 10 to 20 carbon atoms, 12 to 20 carbon atoms, 6 to about 10 carbon atoms or 6 to 8 carbon atoms. Examples of (C₆-C₂₀)aryl groups include phenyl, napthalenyl, azulenyl, biphenylyl, indacenyl, fluorenyl, phenanthrenyl, triphenylenyl, pyrenyl, naphthacenyl, chrysenyl, anthracenyl groups. Examples include substituted phenyl, substituted napthalenyl, substituted azulenyl, substituted biphenylyl, substituted indacenyl, substituted fluorenyl, substituted phenanthrenyl, substituted triphenylenyl, substituted pyrenyl, substituted naphthacenyl, substituted chrysenyl, and substituted anthracenyl groups. Examples also include unsubstituted phenyl, unsubstituted napthalenyl, unsubstituted azulenyl, unsubstituted, biphenylyl, unsubstituted indacenyl, unsubstituted fluorenyl, unsubstituted phenanthrenyl, unsubstituted triphenylenyl, unsubstituted pyrenyl, unsubstituted naphthacenyl, unsubstituted chrysenyl, and unsubstituted anthracenyl groups. Aryl includes phenyl groups and also non-phenyl aryl groups. From these examples, it is clear that the term (C₆-C₂₀)aryl encompasses mono- and polycyclic (C₆-C₂₀)aryl groups, including fused and non-fused polycyclic (C₆-C₂₀)aryl groups.

The term “heteroaryl” as used herein refers to substituted aromatic and unsubstituted aromatic rings containing 3 or more atoms in the ring, of which, one or more is a heteroatom such as, but not limited to, N, O, and S. A heteroaryl can be polycyclic. In some embodiments, heteroaryl groups include 3 to about 20 ring members, whereas other such groups have 3 to about 15 ring members. Representative heteroaryl groups include furanyl, pyridinyl, pyrazinyl, pyrimidinyl, triazinyl, thiophenyl, tetrahydrofuranyl, pyrrolyl, oxazolyl, imidazolyl, triazyolyl, tetrazolyl, benzoxazolinyl, and benzimidazolinyl groups.

In some embodiments, the heteroaryl is a 5-membered heteroaryl. In some embodiments, the heteroaryl is other than pyridine, pyrimidine, pyridazine, pyrazine, or fused derivatives thereof.

The term “alkoxy” as used herein refers to an oxygen atom connected to an alkyl group. The point of substitution to the parent moiety is at the oxygen atom.

The term “aryloxy” as used herein refers to an oxygen atom connected to an aryl group as are defined herein. The point of substitution to the parent moiety is at the oxygen atom.

The term “arylcarbonyl” as used herein refers to a carbonyl (CO) group connected to an aryl group as are defined herein. The point of substitution to the parent moiety is at the carbonyl group.

The terms “halo,” “halogen,” or “halide” group, as used herein, by themselves or as part of another substituent, mean, unless otherwise stated, a fluorine, chlorine, bromine, or iodine atom.

The term “amino” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, alkenyl, alkynyl, alkoxy, aryl, cycloalkyl, heterocyclyl, group or the like.

The term “formyl” as used herein refers to a group containing an aldehyde moiety. The point of substitution to the parent moiety is at the carbonyl group.

The term “alkoxycarbonyl” as used herein refers to a group containing a carbonyl moiety wherein the group is bonded via the carbonyl carbon atom. The carbonyl carbon atom is also bonded to an oxygen atom which is further bonded to an alkyl group. Alkoxycarbonyl also includes the group where a carbonyl carbon atom is also bonded to an oxygen atom which is further bonded to an alkyenyl group. Alkoxycarbonyl also includes the group where a carbonyl carbon atom is also bonded to an oxygen atom which is further bonded to an alkynyl group. in a further case, which is included in the definition of alkoxycarbonyl as the term is defined herein, and is also included in the term “aryloxycarbonyl,” the carbonyl carbon atom is bonded to an oxygen atom which is bonded to an aryl group instead of an alkyl group.

The term “alkylamido” as used herein refers to a group containing a carbonyl moiety wherein the group is bonded via the carbonyl carbon atom. The carbonyl carbon atom is also bonded to a nitrogen group which is bonded to one or more alkyl groups. In a further case, which is also an alkylamido as the term is defined herein, the carbonyl carbon atom is bonded to a nitrogen atom which is bonded to one or more aryl group instead of, or in addition to, the one or more alkyl group. In a further case, which is also an alkylamido as the term is defined herein, the carbonyl carbon atom is bonded to a nitrogen atom which is bonded to one or more alkenyl group instead of, or in addition to, the one or more alkyl and or/aryl group. In a further case, which is also an alkylamido as the term is defined herein, the carbonyl carbon atom is bonded to a nitrogen atom which is bonded to one or more alkynyl group instead of, or in addition to, the one or more alkyl, alkenyl and/or aryl group.

The term “carboxy” as used herein refers to a group containing a carbonyl moiety wherein the group is bonded via the carbonyl carbon atom. The carbonyl carbon atom is also bonded to a hydroxy group or oxygen anion so as to result in a carboxylic acid or carboxylate. Carboxy also includes both the protonated form of the carboxylic acid and the salt form. For example, carboxy can be understood as COOH or CO₂H.

The term “alkylthio” as used herein refers to a sulfur atom connected to an alkyl, alkenyl, or alkynyl group as defined herein. The point of substitution to the parent moiety is at the sulfur atom.

The term “arylthio” as used herein refers to a sulfur atom connected to an aryl group as defined herein. The point of substitution to the parent moiety is at the sulfur atom.

The term “alkylsulfonyl” as used herein refers to a sulfonyl group connected to an alkyl, alkenyl, or alkynyl group as defined herein. The point of substitution to the parent moiety is at the sulfonyl group.

The term “alkylsulfinyl” as used herein refers to a sulfonyl group connected to an alkyl, alkenyl, or alkynyl group as defined herein. The point of substitution to the parent moiety is at the sulfonyl group. The term “amide” linker refers to a —CO—NH- linkage or, alternatively, a —NH—CO- linkage. The nitrogen can be optionally substituted.

The term “oxime” linker refers to a —C═NOH- linkage, a —O—C═NOH- linkage, or a —C═NOH—O- linkage, a —NH—C═NOH- linkage, or a —C═NOH—NH- linkage. The nitrogen or alcohol can be optionally substituted.

The term “carbamate” linker refers to a —O—CO—NH- linkage or a —NH—CO—O- linkage. The nitrogen can be optionally substituted.

The term “sulfonamide” linker refers to a —SO2—NH- linkage or a —NH—SO2- linkage. The nitrogen can be optionally substituted.

The term “carbamide” linker refers to a —NH—CO—NH- linkage or a —NH—CO—NH- linkage. The nitrogens can be optionally substituted.

When the linker is triazole, oxazole, or oxadiazole, the heterocycles can be oriented in any fashion, such that the divalent linkage occurs at, e.g., positions 1 and 2, positions 1 and 3, positions 1 and 4, and any other suitable variation. Moreover, the triazole, oxazole, or oxadiazole can have any suitable orientation of heteroatoms. For example, the triazole can be a 1,2,3-triazole, a 1,2,4-triazole, a 1,3,4-triazole, and the like.

Each of the various substituent groups described herein can be substituted or unsubstituted. The term “substituted” as used herein refers to a group that is substituted with one or more groups (substituents) including, but not limited to, the following groups: deuterium (D), halogen (e.g., F, Cl, Br, and I), R, OR, OC(O)N(R)₂, CN, NO, NO₂, ONO₂, azido, CF₃, OCF₃, methylenedioxy, ethylenedioxy, (C₃-C₂₀)heteroaryl, N(R)₂, Si(R)₃, SR, SOR, SO₂R, SO₂N(R)₂, SO₃R, P(O)(OR)₂, OP(O)(OR)₂, C(O)R, C(O)C(O)R, C(O)CH₂C(O)R, C(S)R, C(O)OR, OC(O)R, C(O)N(R)₂, C(O)N(R)OH, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen, (C₁-C₂₀)alkyl or (C₆-C₂₀)aryl. Substituted also includes a group that is substituted with one or more groups including, but not limited to, the following groups: fluoro, chloro, brorno, iodo, amino, amido, alkyl, alkoxy, alkylamido, alkenyl, alkynyl, alkoxycarbonyl, acyl, formyl, arylcarbonyl, aryloxycarbonyl, aryloxy, carboxy, haloalkyl, hydroxy, cyano, nitroso, nitro, azido, trifluoromethyl, trifluoromethoxy, thio, alkylthio, arylthiol, alkylsulfonyl, alkylsulfinyl, dialkylaminosulfonyl, sulfonic acid, carboxylic acid, dialkylamino and dialkylamido. Where there are two or more adjacent substituents, the substituents can be linked to form a carbocyclic or heterocyclic ring. Such adjacent groups can have a vicinal or germinal relationship, or they can be adjacent on a ring in, e.g., an ortho-arrangement. Each instance of substituted is understood to be independent. For example, a substituted aryl can be substituted with bromo and a substituted heterocycle on the same compound can be substituted with alkyl.

### Exemplary Methods of Identifying Insulin Sensitizers

An assay was used to identify potentiators of insulin-stimulated GLUT4 translocation. In one embodiment, the assay employs Nanobit technology. This technology is based on the recombination of two complementary fragments, HiBiT and LgBiT, of the Nanoluc luciferase. HiBit was fused to an extracellular region of GLUT4 to allow for the rapid quantitation of its the surface density in live cells by adding the reconibinantly produced complementary fragment LgBiT and the luciferase substrate. Since LgBiT is not membrane permeant, only proteins at the plasma membrane are quantified. To measure GLUT4 translocation, a stable C2C12 myoblast cell line was prepared by expressing a HiBiT-tagged mouse GLUT4. A clone was identified in which HiBiT-GLUT4 was localized in storage vesicles, was expressed at low level, and displayed a reliable dynamic range in the insulin-stimulated GLUT4 translocation assay. The Chembridge Diverset library of 49,600 compounds was screened and 1264 hits were identified. A subset of those hits were selected and retested in the presence and absence of insulin. 80 of those molecules significantly enhanced insulin-stimulated GLUT4 translocation but did not promote GLU4 translocation in the absence of insulin.

In order to test the identified insulin sensitizers in an in vivo system, a mouse model was generated in which HiBiT was inserted in the region coding for the first extracellular loop of the endogenous GLUT4 gene using CRISPR/Cas9 technology (HBG4 mouse). GLUT4 was readily detectable using the HiBiT technology in the appropriate tissue of this mouse (e.g., high luminescent signal in lysates of skeletal muscle and adipose tissues, no signal in brain, spleen, liver or pancreas).

Sequence of mGLUT4 in C2C12 HBG4 line (HiBiT and Myc tag were inserted in the first extracellular loop of GLUT4 and mCherry at the end):

(SEQ ID NO: 10)
MPSGFQQIGSDDGEPPRQRVTGTLVLAVFSAVLGSLQFGYNIGVINAPQ
KVIEQSYNATWLGRQGPGRASTPVSGGSVSGWRLFKKISGSSGGSSGRE
AEEQKLISEEDLLKRVAGGPDSIPQGTLTTLWALSVAIFSVGGMISSFL
IGIISQWLGRKRAMLANNVLAVLGGALMGLANAAASYEILILGRFLIGA
YSGLTSGLVPMYVGEIAPTHLRGALGTLNQLAIVIGILVAQVLGLESML
GTATLWPLLLALTVLPALLQLILLPFCPESPRYLYIIRNLEGPARKSLK
RLTGWADVSDALAELKDEKRKLERERPMSLLQLLGSRTHRQPLIIAVVL
QLSQQLSGINAVFYYSTSIFESAGVGQPAYATIGAGVVNTVFTLVSVLL
VERAGRRTLHLLGLAGMCGCAILMTVALLLLERVPAMSYVSIVAIFGFV
AFFEIGPGPIPWFIVAELFSQGPRPAAMAVAGFSNWTCNFIVGMGFQYV
ADAMGPYVFLLFAVLLLGFFIFTFLKVPETRGRTFDQISAAFRRTPSLL
EQEVKPSTELEYLGPDENDGDPPEFTTMVSKGEEDNMAIIKEFMRFKVH
MEGSVNGHEFEIEGEGEGRPYEGTQTAKLKVTKGGPLPFAWDILSPQFM
YGSKAYVKHPADIPDYLKLSFPEGFKWERVMNFEDGGVVTVTQDSSLQD
GEFIYKVKLRGTNFPSDGPVMQKKTMGWEASSERMYPEDGALKGEIKQR
LKLKDGGHYDAEVKTTYKAKKPVQLPGAYNVNIKLDITSHNEDYTIVEQ
YERAEGRHSTGGMDELYK(mCherry)

or a polypeptide sequence having at least 80%, 85%, 90%, 95%, 97%, 98%, or 99% amino acid sequence identity thereto.

Sequence of HiBiT-GLUT4 in the mouse model (HiBiT sequence (VSGWRLFKKIS; SEQ ID NO:20) surrounded with linker (GSSGGSSG; SEQ ID NO:21) was inserted in the endogenous GLUT4 gene using CRISPR/Cas9 technology with a gRNA sequence CTGGGTAGGCAAGGTCCTGG (SEQ. ID NO:22) targeting exon 3 in the coding sequence of the first extracellular loop. The translated GLUT4 sequence resulting is:

(SEQ ID NO: 11)
MPSGFQQIGSDDGEPPRQRVTGTLVLAVFSAVLGSLQFGYNIGVINAPQ
KVIEQSYNATWIGRQGPGSSGGSSGVSGWRLFKKISGSSGGSSGGGPDS
IPQGTLTTLWALSVAIFSVGGMISSFLIGIISQWLGRKRAMLANNVLAV
LGGALMGLANAAASYEILILGRFLIGAYSGLTSGLVPMYVGEIAPTHLR
GALGTINQLAIVIGILVAQVLGLESMLGTATLWPLLLALTVLPALLQLI
LLPFCPESPRYLYIIRNLEGPARKSLKRLTGWADVSDALAELKDEKRKL
ERERPMSLLQLLGSRTHRQPLIIAVVLQLSQQLSGINAVFYYSTSIFES
AGVGQPAYATIGAGVVNTVFTLVSVLLVERAGRRTLHLLGLAGMCGCAI
LMTVALLLLERVPAMSYVSIVAIFGFVAFFEIGPGPIPWFIVAELFSQG
PRPAAMAVAGFSNWTCNFIVGMGFQYVADAMGPYVFLLFAVLLLGFFIF
TFLKVPETRGRTFDQISAAFRRTPSLLEQEVKPSTELEYLGPDEND

or a polypeptide sequence having at least 80%, 85%, 90%, 95%, 97%, 98%, or 99% amino acid sequence identity thereto.

An exemplary human GLUT4 sequence for use in the assays includes but is not limited to:

(SEQ ID NO: 1)
mpsgfqqigs edgeppqqrv tgtlvlavfs avlgslqfgy
nigvinapqk vieqsynetw lgrqgpegps sippgtlttl
walsvaifsv ggmissflig iisqwlgrkr amlvnnvlav
lggslmglan aaasyemlil grfligaysg ltsglvpmyv
geiapthlrg algtlnqlai vigiliaqvl glesligtas
lwplllgltv lpallqlvll pfcpespryl yiignlegpa
rkslkrltgw advsgvlael kdekrklere rplsllqllg
srthrqplii avvlqlsqql sginavfyys tsifetagvg
qpayatigag vvntvftlvs vllveragrr tlhllglagm
cgcailmtva llllervpam syvsivaifg fvaffeigpg
pipwfivael fsqgprpaam avagfsnwts nfiigmgfqy
vaeamgpyvf llfavlllgf fiftflrvpe trgrtfdqis
aafhrtpsll eqevkpstel eylgpdend

or a polypeptide sequence having at least 80%, 85%, 90%, 95%, 97%, 98%, or 99% amino acid sequence identity thereto. A cell line, e.g., human cell line, expressing hGLUT4 similar to C2C12 HBG4 line expressing mGLUT4 may be used.

For example, a compound that may potentiate insulin-stimulated GLUT4 translocation in vitro or result in an increase in GLUT4 membrane localization in skeletal muscle and adipose tissues in vivo, has the structure:

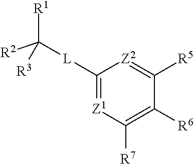

wherein

each of R¹, R², and R3 is independently alkyl, aryl, or heteroaryl, or two or three of R1, R², and R³ taken together with the carbon to which they attach form a cycloalkyl ring;

L is a linker selected from amide, oxime, carbamate, carbamide; triazole, oxazole, oxadiazole, or sulfonamide;

each of Z¹ and Z² is independently CR⁸, CR⁹, or N;

each of R⁵, R⁵, R⁷, R⁸, and R⁹, if present, is independently H, fluoro; chloro, bromo, iodo, amino, amide, alkyl, alkoxy, alkylamino, alkylthio, alkylamido, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring; and

when Z¹ and Z² are CR⁸ or CR⁹, or when R¹, R², and R3 taken together are adamantyl, then at least one two of R⁵, R⁶, R⁷, R⁸, and R⁹ is other than hydrogen, and at least one of R⁵, R⁶, R⁷, R⁸, and R⁹ other than fluoro, chloro, promo, and iodo.

In one embodiment, a compound that may potentiate insulin-stimulated GLUT4 translocation in vitro or result in an increase in GLUT4 membrane localization in skeletal muscle and adipose tissues in vivo, has the structure:

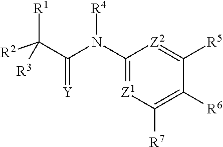

wherein

each of R¹, R², and R3 is independently H, alkyl, aryl, or heteroaryl, or two or three of R1, R², and R³ taken together with the carbon to which they attach form a cycloalkyl ring;

L is a linker selected from amide, oxime, carbamate, sulfonamide, or carbamide;

Y is O, S, or NR10,

each of R4 and R10 is independently hydrogen, alkyl, aryl, hydroxyl;

each of Z¹ and Z² is independently CR⁸, CR⁹, or N;

each of R⁵, R⁶, R⁷, R⁸, and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarbonyl, arylamido; aryloxycarbonyl, hydroxy, amino, cyano, nitro, azide, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring, and

when Z¹ and Z² are CR⁸ or CR⁹, or when R¹, R², and R3 taken together are adamantyl, then at least one two of R⁵, R⁶, R⁷, R⁸, and R⁹ is other than hydrogen, and at least one of R⁵, R⁶, R⁷, R⁸, and R⁹ other than fluoro, chloro, bromo, and iodo.

### Exemplary Compounds

In one embodiment, a compound is provided that has the structure:

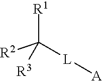

wherein

each of R1, R2, and R3 is independently H, alkyl, aryl, or heteroaryl, or two or three of R1, R2, and R3 taken together with the carbon to which they attach form a cycloalkyl ring;

L is a linker selected from amide, oxime, carbamate, carbamide, triazole, oxazole, oxadiazole, or sulfonamide; and

A is aryl or heteroaryl.

In one embodiment, A is substituted aryl, substituted heteroaryl, or unsubstituted heteroaryl.

In one embodiment, A is substituted with one, two, or three chloro.

In one embodiment, A is a heteroaryl.

In one embodiment, A is pyridine, pyrimidine, pytidazine, pyrazine, quinoline, isoquinoline, or naphthylene.

In one embodiment, A is a dichlorophenyl, optionally substituted with one or more additional substituent.

In one embodiment, A is other than phenyl.

In one embodiment, A is 2-pyridinyl, 2-pyrimidinyl, or 3-isoquinolyl.

In one embodiment, A is a 6-membered ring has a halogen para to the linkage to L.

In one embodiment, A is a 6-membered ring having a 4-halo, 5-halo, or both.

In one embodiment, A is a 6-membered ring having a 4-chloro, 5-chloro, or both.

In one embodiment, A is a 6-membered heteroaryl having a 4-chloro, 5-chloro, or both.

In one embodiment, the compound has the structure:

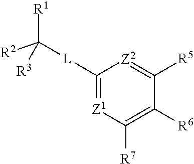

wherein

each of R¹, R², and R3 is independently H, alkyl, aryl, or heteroaryl, or two or three of R1, R², and R³ taken together with the carbon to which they attach form a cycloalkyl ring;

L is a linker selected from amide, oxime, carbamate; sulfonamide, or carbamide;

each of Z¹ and Z² is independently CR⁸, CR^{9,} or N;

each of R⁵, R⁶, R⁷, R⁸, and R⁹, if present, is independently H, fluoro, chloro, bromo, iodo, amino, amido, alkyl, alkoxy, alkylamino, alkylthio, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring; and

when Z¹ and Z² are other than N, then at least one of R⁵, R⁶, R⁷, R⁸, and R⁹ is other than hydrogen.

In one embodiment, the compound has the structure:

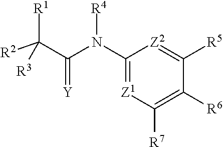

wherein

each of R¹, R², and R3 is independently H, alkyl, aryl, or heteroaryl, or two or three of R1, R², and R³ taken together with the carbon to which they attach form a cycloalkyl ring;

L is a linker selected from amide, oxime, carbamate, sulfonamide, or carbamide;

Y is O, S, or NR10,

each of R4 and R10 is independently hydrogen, alkyl, aryl, hydroxyl;

each of Z¹ and Z² is independently CR⁸, CR⁹, or N;

each of R⁵, R⁶, R⁷, R⁸, and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸ and R⁹ taken together with the carbons to which they attach form an aryl or heteroaryl ring; and

when Z¹ and Z² are other than N, then at least one two of R⁵, R⁶, R⁷, R⁸, and R⁹ is other than hydrogen.

### Methods of Using a Compound of Formula (I)-(III)

Type 2 diabetes (T2D) is major cause of mortality and morbidity in developed countries. The key manifestations of his disease are insulin resistance (IR), hyperglycemia and hyperinsulinemia. In healthy people, insulin is secreted by the endocrine pancreas in response to elevated plasma glucose levels. Insulin promotes the clearance of glucose from the circulation by storing it in skeletal muscle and adipose tissue. The mechanism underlying glucose clearance from the circulation involves insulin signaling in muscle and fat cells leading to a series of intracellular events that trigger the translocation of vesicles containing the glucose transporter 4 (GLUT4) to the cell surface. In individuals with IR or T2D, insulin-stimulated GLUT4 translocation is impaired, resulting in inefficient transport of glucose through the plasma membrane and hyperglycemia. Chronic hyperglycemic condition increases the risk of cardiovascular disease, stroke, neuropathy, and death. Identifying insulin sensitizers to restore insulin-stimulated GLUT4 translocation in diabetic patients is needed, however, their identification has been challenging due to the lack of a GLUT4 translocation assay amenable to high throughput screening (HTS).

In one embodiment, a method for preventing, inhibiting or treating type II diabetes in a mammal is provided.

In one embodiment, a method providing for or enhancing glycemic control in a mammal is provided.

In one embodiment, a method for enhancing insulin-stimulated GLUT4 translocation in a mammal is provided.

In one embodiment, a method for preventing, inhibiting or treating insulin resistance in a mammal is provided.

In one embodiment, the method includes administering an effective amount of a compound having the structure (I):

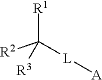

wherein

each of R1, R2, and R3 is independently H, alkyl, aryl, or heteroaryl, or two or three of R1, R2, and R3 taken together with the carbon to which they attach form a cycloalkyl ring;

L is a linker selected from amide, oxime, carbamate, carbamide, triazole, oxazole, oxadiazole, or sulfonamide; and

A is aryl or heteroaryl.

In one embodiment, A is substituted aryl, substituted heteroaryl, or unsubstituted heteroaryl. In one embodiment, A is substituted with one, two, or three chloro. In one embodiment, A is a heteroaryl. In one embodiment, A is pyridine, pyrimidine, pyridazine, pyrazine, quinoline, isoquinoline, or naphthylene. In one embodiment, A is a dichlorophenyl, optionally substituted with one or more additional substituent. In one embodiment, A is other than phenyl. In one embodiment, A is 2-pyridinyl, 2-pyrimidinyl, or 3-isoquinolyl. In one embodiment, A is a 6-membered ring has a halogen para to the linkage to L. In one embodiment, A is a 6-membered ring having a 4-halo, 5-halo, or both. In one embodiment,

A is a 6-membered ring having a 4-chloro, 5-chloro, or both. In one embodiment, A is a 6-membered heteroaryl having a 4-chloro, 5-chloro, or both.

In one embodiment, the method includes administering an effective amount of a compound having the structure (II):

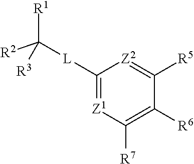

wherein

each of R¹, R², and R3 is independently alkyl, aryl, or heteroaryl, or two or three of R1, R², and R³ taken together with the carbon to which they attach form a cycloalkyl ring;

L is a linker selected from amide, oxime, carbamate, sulfonamide, or carbamide;

each of Z¹ and Z² is independently CR⁸, CR⁹, or N;

each of R⁵, R⁶, R⁷, R⁸, and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring; and

when Z¹ and Z² are other than N, then at least one of R⁵, R⁶, R⁷, R⁸, and R⁹ is other than hydrogen.

In one embodiment, the compound has the structure (III):

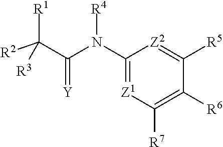

wherein

each of R¹, R², and R3 is independently H, alkyl, aryl, or heteroaryl; or two or three of R1, R², and R³ taken together with the carbon to which they attach form a cycloalkyl ring;

L is a linker selected from amide, oxime, carbamate, sulfonamide, or carbamide;

Y is O, S, or NR10;

each of R4 and R10 is independently hydrogen, alkyl, aryl, hydroxyl;

each of Z¹ and Z² is independently CR⁸, CR⁹, or N;

each of R⁵, R⁶, R⁷, R⁸, and R⁹, if present, is independently H, fluoro, chloro, bromo, iodo, amino, amino, alkyl, alkoxy, alkylamino, alkylthio, alkylamido, formyl, acyl, alkoxycarbonyl, acyloxy, aryloxy, arylamino, arylthio, arylcarbonyl, arylamido; aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring; and

when Z¹ and Z² are other than N, then at least one two of R⁵, R⁶, R⁷, R⁸, and R⁹ is other than hydrogen.

In one embodiment, the method includes administering an effective amount of a compound having the structure:

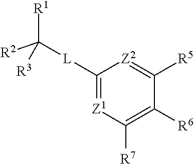

wherein

each of R¹, R², and R3 is independently H, alkyl, aryl, or heteroaryl, or two or three of R1, R², and R³ taken together with the carbon to which they attach form a cycloalkyl ring;

L is a linker selected from amide, oxime, carbamate, carbamide, triazole, oxazole, oxadiazole, or sulfonamide;

each of Z¹ and Z² is independently CR⁸, CR⁹, or N;

each of R⁵, R⁶, R⁷, R⁸, and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring; and

when Z¹ and Z² are CR⁸ or CR⁹, or when R¹, R², and R3 taken together are adamantyl, then at least one two of R⁵, R⁶, R⁷, R⁸, and R⁹ is other than hydrogen, and at least one of R⁵, R⁶, R⁷, R⁸, and R⁹ other than fluoro, chloro, bromo, and iodo.

In one embodiment, the method includes administering an effective amount of a compound having the structure:

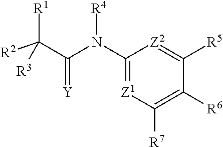

wherein

each of R¹, R², and R3 is independently H, alkyl, aryl, or heteroaryl, or two or three of R¹, R², and R³ taken together with the carbon to which they attach form a cycloalkyl ring;

L is a linker selected from amide, oxime, carbamate, sulfonamide, or carbamide;

Y is O, S, or NR10,

each of R4 and R10 is independently hydrogen, alkyl, aryl, hydroxyl;

each of Z¹ and Z² is independently CR⁸, CR⁹, or N;

each of R⁵, R⁶, R⁷, R⁸, and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarbonyl, atylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring; and

when Z¹ and Z² are CR⁸ or CR⁹, or when R¹, R², and R3 taken together are adamantyl, then at least one two of R⁵, R⁶, R⁷, R⁸, and R⁹ is other than hydrogen, and at least one of R⁵, R⁶, R⁷, R⁸, and R⁹ other than fluoro, chloro, bromo, and iodo.

In one embodiment of structure (III), Y is O, or N—OH.

In one embodiment, R¹, R², and R3 taken together with the carbon to which they attach form a cycloalkyl.

In one embodiment, R¹, R², and R3 taken together with the carbon to which they attach form an adamantyl ring.

In one embodiment, two of R¹, R², and R3 taken together with the carbon to which they attach form a cycloalkyl.

In one embodiment, two of R¹, R², and R3 taken together with the carbon to which they attach form a cyclohexane or tetrahydropyran.

In one embodiment, at least one of Z¹ and Z² is N.

In one embodiment, Z¹ and Z² are independently CR⁸ or CR⁹ and at least two of R⁵, R⁶, R⁷, R⁸, and R⁹ are chloro and at least one of R⁵, R⁶, R⁸, and R⁹ are other than chloro and other than hydrogen.

In one embodiment, at least two of R⁵, R⁶, R⁷, R⁸, and R⁹ is chloro, fluoro, bromo, or iodo.

In one embodiment, R⁶ is chloro, fluoro, brorno, or iodo.

In one embodiment, one of R⁵ and R⁷ is methyl, chloro, flouoro, or bromo.

In one embodiment, the compound has the structure:

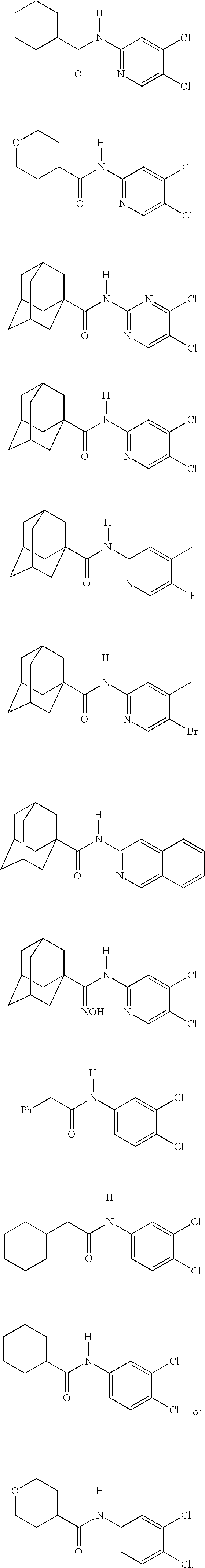

In one embodiment, the compound has the structure:

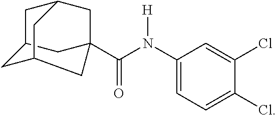 [SMILES: O=C(Nc1ccc(Cl)c(Cl)c1)C12CC3CC(CC(C3)C1)C2]

### Pharmaceutical Compositions

The disclosure provides a composition comprising, consisting essentially of, or consisting of a compound disclosed herein and optionally a pharmaceutically acceptable (e.g., physiologically acceptable) carrier. In one embodiment, additional components can be included that do not materially affect the composition (e.g., adjuvants, buffers, stabilizers, anti-inflammatory agents, solubilizers, preservatives, etc.). In one embodiment, when the composition consists of the compound disclosed herein and optionally the pharmaceutically acceptable carrier, the composition does not comprise any additional components. Any suitable carrier can be used within the context of the invention, and such carriers are well known in the art. The choice of carrier will be determined, in part, by the particular site to which the composition may be administered and the particular method used to administer the composition. The composition may be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, Pa. (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile: powders, granules, and tablets of the kind previously described. In one embodiment, the carrier is a buffered saline solution. In one embodiment, the compound is administered in a composition formulated to protect the compound from damage prior to administration. In addition, one of ordinary skill in the art will appreciate that the compound(s) can be present in a composition with other biologically-active agents.

Injectable depot forms are envisioned including those having biodegradable polymers such as polylactide-polyglycolide Depending on the ratio of compound to polymer, and the nature of the particular polymer employed, the rate of compound release can be controlled. Examples of other biodegradable polymers include poly(orthoesters) and polyanhydrides. Depot injectable formulations are also prepared by entrapping the compound optionally in a complex with a polymer in liposomes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration. The composition also can be administered in the form of sustained-release formulations (see. e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or apolylactic-glycolic acid.

The dose of the compound in the composition administered to the mammal will depend on a number of factors, including the size (mass) of the mammal, the extent of any side-effects, the particular route of administration, and the like. In one embodiment, the method comprises administering a “therapeutically effective amount” of the composition. A “therapeutically effective amount” refers to an amount effective, at dosages and for periods of time necessary, to achieve a desired therapeutic result, e.g., enhancing an immune response. The therapeutically effective amount may vary according to factors such as the extent of the disease or disorder, age, sex, and weight of the individual, and the ability of the nanoparticles to elicit a desired response in the individual. One of ordinary skill in the art can readily determine an appropriate dose range in a patient having a particular disease or disorder, or in need of eliciting a desried response, based on these and other factors that are well known in the art.

The present disclosure provides pharmaceutically acceptable compositions which comprise an amount of the nanoparticles as described above.

### Routes of Administration, Dosages and Dosage Forms

Administration may be continuous or intermittent, depending, for example, upon the recipient's physiological condition, and other factors known to skilled practitioners. The administration may be essentially continuous over a preselected period of time, may be in a series of spaced doses, or may be a single dose. Both local administration and systemic administration, e.g., intravenous or oral, are envisioned. In one embodiment, compositions may be subcutaneously, intramuscularly, intradermally, or intravascularly delivered.

One or more suitable unit dosage forms comprising the compound, which may optionally be formulated for sustained release, can be administered by a variety of routes including local, e.g., intrathecal, oral, or parenteral, including by rectal, buccal, vaginal and sublingual, transdermal, subcutaneous, intravenous, intramuscular, intraperitoneal, intrathoracic, or intrapul monary routes. The formulations may, where appropriate, be conveniently presented in discrete unit dosage forms and may be prepared by any of the methods well known to pharmacy. Such methods may include the step of bringing into association the nanoparticles with liquid carriers, solid matrices, semi-solid carriers, finely divided solid carriers or combinations thereof, and then, if necessary, introducing or shaping the product into the desired delivery system.

The amount of the compound administered to achieve a particular outcome will vary depending on various factors including, but not limited to the condition, patient specific parameters, e.g., height, weight and age, and whether prevention or treatment, is to be achieved.

The compounds may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof

The compounds may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The compounds can be provided in a dosage form containing an amount effective in one or multiple doses, e.g., administered in dosages of at least about 0.0001 mg/kg to about 1 mg/kg, of at least about 0.001 mg/kg to about 0.5 mg/kg, at least about 0.01 mg/kg to about 0.25 mg/kg, at least about 0.01 mg/kg to about 0.25 mg/kg of body weight, at least about 0.1 mg/kg to about 25 mg/kg of body weight, at least about 1 mg/kg to about 250 mg/kg of body weight, at least about 10 mg/kg to about 500 mg/kg of body weight, at least about 0.1 g/kg to about 0.5 g/kg of body weight, or at least about 0.5 g/kg to about 2 g/kg of body weight, although other dosages may provide beneficial results. The amount administered will vary depending on various factors including, but not limited to, the disease, the weight, the physical condition, the health, and/or the age of the mammal. Such factors can be readily determined by the clinician employing animal models or other test systems that are available in the art. As noted, the exact dose to be administered is determined by the attending clinician but may be in 1 mL phosphate buffered saline. In one embodiment, from 0.0001 to 1 mg or more, e.g., up to 1 g, in individual or divided doses, e.g., from 0.001 to 0.5 mg, or 0.01 to 0.1 mg, of nanoparticles can be administered.

Pharmaceutical formulations can be prepared by procedures known in the art using well known and readily available ingredients. For example, the compound can be formulated with common excipients, diluents, or carriers, and formed into tablets, capsules, suspensions, powders, and the like. The compound(s) can also be formulated as elixirs or solutions appropriate for parenteral administration, for instance, by intramuscular, subcutaneous or intravenous routes.

The pharmaceutical formulations can also take the form of an aqueous or anhydrous solution, e.g., a lyophilized formulation, or dispersion, or alternatively the form of an emulsion or suspension.

In one embodiment, the compound(s) may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the composition is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the nanoparticles and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler. p For intra-nasal administration, the composition may be administered via nose drops, a liquid spray, such as via a plastic bottle atomizer or metered-dose inhaler. Typical atomizers are the Mistometer (Wintrop) and the Medihaler (Riker).

The local delivery of the composition can also be by a variety of techniques which administer the composition at or near the site of disease, e.g., using a catheter or needle. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### Subjects

The subject may be any animal, including a human and non-human animals. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. The subject may also be livestock such as, cattle, swine, sheep, poultry, and horses, or pets, such as dogs and cats.

The subject is generally diagnosed with the condition of the subject invention by skilled artisans, such as a medical practitioner. is The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods of the invention may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any body height, body weight, or any organ or body part size or shape.

### Exemplary Embodiments

In one embodiment, a method to sensitize a mammal to insulin is provided which includes comprising administering to the mammal an effective amount of a compound having the structure (I):

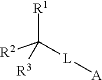

wherein

each of R1, R2, and R3 is independently H, alkyl, aryl, or heteroaryl, or two or three of R1, R2, and R3 taken together with the carbon to which they attach form a cycloalkyl ring; L is a linker selected from amide, oxime, carbamate, carbamide, triazole, oxazole, oxadiazole, or sulfonamide; and A is aryl or heteroaryl. In one embodiment, A is substituted aryl, substituted heteroaryl, or unsubstituted heteroaryl. In one embodiment, A is substituted with one, two, or three chloro. In one embodiment, A is a heteroaryl. In one embodiment, A is pyridine, pyrimidine, pyridazine, pyrazine, quinoline, isoquinoline, or naphthylene. In one embodiment, A is a dichlorophenyl, optionally substituted with one or more additional substituent. In one embodiment, A is other than phenyl. In one embodiment, A is 2-pyridinyl, 2-pyrimidinyl, or 3-isoquinolyl. In one embodiment, A is a 6-membered ring has a halogen para to the linkage to L. In one embodiment, A is a 6-membered ring having a 4-halo, 5-halo, or both. In one embodiment, A is a 6-membered ring having a 4-chloro, 5-chloro, or both. In one embodiment, A is a 6-membered heteroaryl having a 4-chloro, 5-chloro, or both. In one embodiment,

the compound has structure (II):

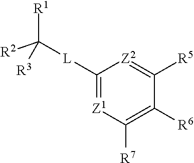

wherein each of R¹, R², and R3 is independently H, alkyl, aryl, or heteroaryl, or two or three of R1, R², and R³ taken together with the carbon to which they attach form a cycloalkyl ring; L is a linker selected from amide, oxime, carbamate, sulfonamide, or carbamide; each of Z¹ and Z² is independently CR⁸, CR⁹, or N; each of R⁵, R⁶, R⁷, R⁸, and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring, and when Z¹ and Z² are other than N, then at least one of R⁵, R⁶, R⁷, R⁸, and R⁹ is other than hydrogen. In one embodiment, the compound has structure (III):

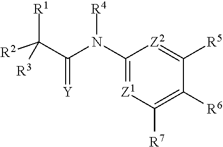

wherein each of R¹, R², and R³ is independently H, alkyl, aryl, or heteroaryl, or two or three of R1, R², and R³ taken together with the carbon to which they attach form a cycloalkyl ring; L is a linker selected from amide, oxime, carbamate, sulfonamide, or carbamide; Y is O, S, or NR10; each of R4 and R10 is independently hydrogen, alkyl, aryl, hydroxyl; each of Z¹ and Z² is independently CR⁸, CR⁹, or N; each of R⁵, R⁶, R⁷, R⁸, and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring, and when Z¹ and Z² are other than N, then at least one two of R⁵, R⁶, R⁷, R⁸, and R⁹ is other than hydrogen. In one embodiment, Y is O, or N—OH. In one embodiment, three of R¹, R², and R³ taken together with the carbon to which they attach form a cycloalkyl. In one embodiment, R¹, R², and R3 taken together with the carbon to which they attach form an adamantyl ring. In one embodiment, two of R¹, R², and R3 taken together with the carbon to which they attach form a cycloalkyl. In one embodiment, two of R¹, R², and R3 taken together with the carbon to which they attach form a cyclohexane or tetrahydropyran. In one embodiment, at least one of Z¹ and Z² is N. In one embodiment, Z¹ and Z² are independently CR⁸ or CR⁹ and at least two of R⁵, R⁶, R⁷, R⁸, and R⁹ are chloro and at least one of R⁵, R⁶, R⁷, R⁸, and R⁹ are other than chloro and other than hydrogen. In one embodiment, at least two of R⁵, R⁵, R⁷, R⁸, and R⁹ is chloro, fluoro, bromo, or iodo. In one embodiment, R⁶ is chloro, fluoro, bromo, or iodo. In one embodiment, one of R⁵ and R⁷ is methyl, chloro, flouoro, or bromo. In one embodiment, the compound has the structure:

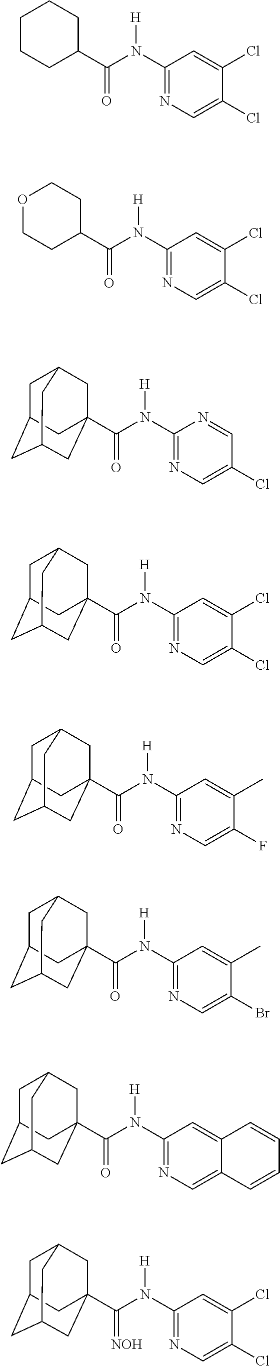

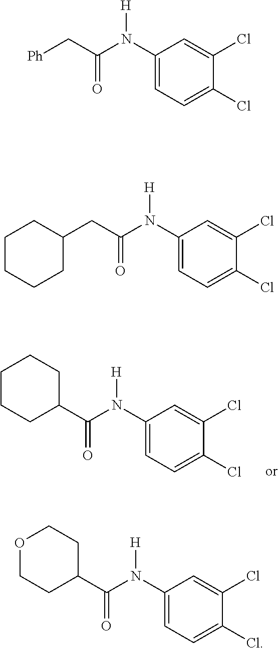 [SMILES: O=C(Cc1ccccc1)Nc1ccc(Cl)c(Cl)c1 ; O=C(CC1CCCCC1)Nc1ccc(Cl)c(Cl)c1 ; O=C(Nc1ccc(Cl)c(Cl)c1)C1CCCCC1 ; O=C(Nc1ccc(Cl)c(Cl)c1)C1CCOCC1]

In one embodiment, the compound has the structure:

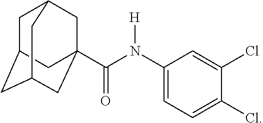 [SMILES: O=C(Nc1ccc(Cl)c(Cl)c1)C12CC3CC(CC(C3)C1)C2]

In one embodiment, the mammal is a human. In one embodiment, the compound is systemically administered, In one embodiment, the compound is orally administered. In one embodiment, the composition is a sustained release dosage form.

Further provided is a compound haying the structure (II):

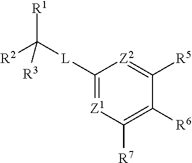

wherein each of R¹, R², and R³ is independently H, alkyl, aryl, or heteroaryl, or two or three of R1, R², and R³ taken together with the carbon to which they attach form a cycloalkyl ring; is a linker selected from amide, oxime, carbamate, carbamide, triazole, oxazole, oxadiazole, or sulfonamide; each of Z¹ and Z² is independently CR⁸, CR⁹, or N; each of R⁵, R⁶, R⁷, R⁸, and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring; and when Z¹ and Z² are CR⁸ or CR⁹, or when R¹, R², and R3 taken together are adamantyl, then at least one two of R⁵, R⁶, R⁷, R⁸, and R⁹ is other than hydrogen, and at least one of R⁵, R⁶, R⁷, R⁸, and R⁹ other than fluoro, chloro, bromo, and iodo. In one embodiment, the compound has structure (III):

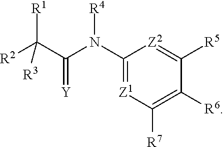

wherein each of R¹, R², and R3 is independently H, alkyl, aryl, or heteroaryl, or two or three of R1, R², and R³ taken together with the carbon to which they attach form a cycloalkyl ring; L is a linker selected from amide, oxime, carbamate, sulfonamide, or carbamide; Y is O, S, or NR10; each of R4 and R10 is independently hydrogen, alkyl, aryl, hydroxyl; each of Z¹ and Z² is independently CR⁸, CR⁹, or N; each of R⁵, R⁶, R⁷, R⁸, and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring; and when Z¹ and Z² are CR⁸ or CR⁹, or when R¹, R², and R3 taken together are adamantyl, then at least one two of R⁵, R⁶, R⁷, R⁸, and R⁹ is other than hydrogen, and at least one of R⁵, R⁶, R⁷, R⁸, and R⁹ other than fluoro, chloro, bromo, and iodo. In one embodiment, Y is O, or N—OH. In one embodiment, three of R¹, R², and R3 taken together with the carbon to which they attach form a cycloalkyl. In one embodiment, R¹, R², and R3 taken together with the carbon to which they attach form an adamantyl ring. In one embodiment, two of R¹, R², and R3 taken together with the carbon to which they attach form a cycloalkyl. In one embodiment, two of R¹, R², and R3 taken together with the carbon to which they attach form a cyclohexane or tetrahydropyran. In one embodiment, at least one of Z¹ and Z² is N. In one embodiment, Z¹ and Z² are independently CR⁸ or CR⁹ and at least two of R⁵, R⁶, R⁷, R⁸, and R⁹ are chloro and at least one of R⁵, R⁶, R⁷, R⁸, and R⁹ are other than chloro and other than hydrogen. In one embodiment, at least two of R⁵, R⁵, R⁷, R⁸, and R⁹ is chloro, fluoro, bromo, or iodo. In one embodiment, R⁶ is chloro, fluoro, bromo, or iodo. In one embodiment, one of R⁵ and R⁷ is methyl, chloro, flouoro, or bromo. In one embodiment, the compound has the structure:

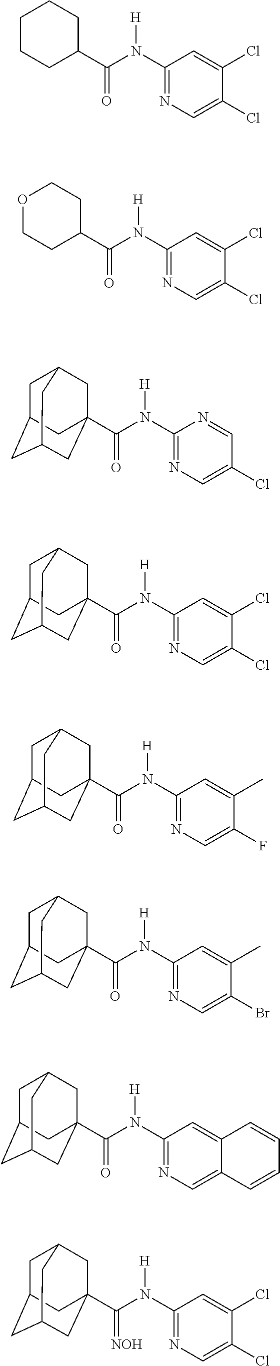

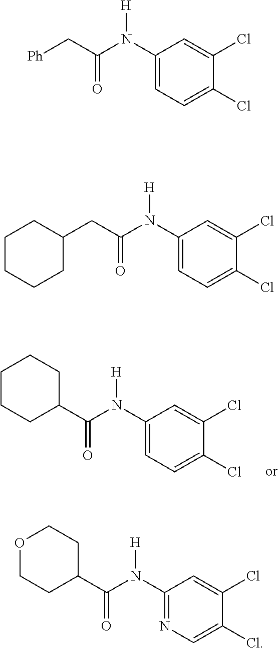 [SMILES: O=C(Cc1ccccc1)Nc1ccc(Cl)c(Cl)c1 ; O=C(CC1CCCCC1)Nc1ccc(Cl)c(Cl)c1 ; O=C(Nc1ccc(Cl)c(Cl)c1)C1CCCCC1 ; O=C(Nc1cc(Cl)c(Cl)cn1)C1CCOCC1]

In one embodiment, the compound has the structure:

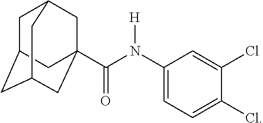 [SMILES: O=C(Nc1ccc(Cl)c(Cl)c1)C12CC3CC(CC(C3)C1)C2]

Also provided is an in vitro method to screen for insulin sensitizers, comprising: contacting one or more test compounds with isolated mammalian cells comprising an expression cassette encoding a fusion polypeptide having at least 80% amino acid sequence identity to GLUT4 sequences in SEQ ID NO:1, 10 or 11 in the presence of insulin, wherein the fusion polypeptide comprises at least one tag; and detecting or determining whether the polypeptide has enhanced translocation to the plasma membrane of the mammalian cells in the presence of the one or more test compounds and insulin relative to corresponding mammalian cells contacted with the one or more test compound. in the absence of insulin. In one embodiment, the cells are murine cells. In one embodiment, the cells are human cells. In one embodiment, the at least one tag is inserted into an extracellular domain of GLUT4. In one embodiment, the at least one tag is inserted into an intracellular domain of GLUT4. In one embodiment, the at least one tag is optically detectable. In one embodiment, the location of the polypeptide is enzymatically detectable.

A method of using a non-human recombinant mammalian model to screen for insulin sensitizers is provided. In one embodiment, one or more test compounds are contacted with a non-human recombinant mammal expressing a fusion polypeptide having at least 80% amino acid sequence identity to GLUT4 sequences in SEQ ID NO:1, 10 or 11 in the presence of insulin, wherein the fusion polypeptide

comprises at least one tag; and detecting or determining whether the polypeptide has enhanced translocation to the plasma membrane relative to a corresponding non
human recombinant mammal contacted with the one or more test compounds in the absence of insulin. In one embodiment, the mammal is a rodent, e.g., a rat, hamster, guinea pig, or mouse, or a ferret or rabbit. In one embodiment, the at least one tag is inserted into an extracellular domain of GLUT4. In one embodiment, the at least one tag is inserted into an intracellular domain of GLUT4. In one embodiment,
the genome of the mammal is modified to yield a sequence encoding the fusion polypeptide.

Further provided is a non-human recombinant mammal expressing a fusion polypeptide having at least 80% amino acid sequence identity to GLUT4 sequences in SEQ ID NO:1, 10 or 11, wherein the fusion polypeptide comprises at least one tag. In one embodiment, the non-human recombinant mammal is a rodent. In one embodiment, the at least one tag is inserted into an extracellular domain of GLUT4.

The invention will be described by the following non-limiting examples.

### EXAMPLE 1

A luminescence based HTS assay was developed that allows live monitoring of GLUT4 translocation in mammalian cells. Following validation of the assay, a library of 50,000 compounds was screened to identify molecules that enhance insulin-stimulated GLUT4 translocation. Hits were validated and characterized pharmacologically to prioritize compounds that enhance insulin action but do not stimulate GLUT4 translocation in the absence of insulin. Based on results from those experiments, C3 was selected for further development. Using structure activity relationship (SAR), derivatives of C3 were prepared that had improved solubility and efficacy. C3 and derivatives thereof significantly enhanced insulin-stimulated GLUT4 translocation and glucose uptake in primary adipocytes, Additionally, C3 administration drastically improved insulin sensitivity and glucose tolerance in both lean and diet-induced obese mice. Whereas the efficacy of C3 in vivo was comparable to FDA approved TZDs, C3 was fast acting and did not activate PPARg (the TZD target), consequently C3 is likely to be safer. Such molecule has a tremendous clinical potential whereby restoring insulin sensitivity it may results in a reversal of THD and a decrease risk of occurrence of associated morbidities,

Development of a HTS assay for real-time measurement of GLUT4 translocation. To measure GLUT4 translocation in a homogenous and HIS amenable manner, NanoBit technology was used. This technology relies on the rapid recombination of 2 fragments of the Nanoluc luciferase (HiBit and LgBiT) with high affinity for each other. Fragments have no enzymatic activity on their own but generate a bright luminescent signal when recombined. A C2C12 myoblast cell line was prepared that stably expressed HiBiT-GLUT4 (HBG4), in which the small 11 amino acid HiBiT fragment was inserted into the first extracellular loop of GLUT4. A C-terminal mCherry was also added for microscopic detection. The C2C12 line was chosen as an in vitro model because it is derived from skeletal myoblasts and translocate GLUT4 in response to insulin. Detection of plasma membrane density of HBG4 is achieved by adding recombinant LgBit and substrate to live cells. The assay can be conducted as an endpoint where cells are incubated with insulin before measuring luminescence or as a kinetic assay by adding LgBiT and substrate at the beginning of the experiment and monitor real-time insulin-stimulated GLUT4 translocation. In both formats the assay displayed an insulin-concentration dependent GLUT4 translocation with an unprecedented dynamic range.

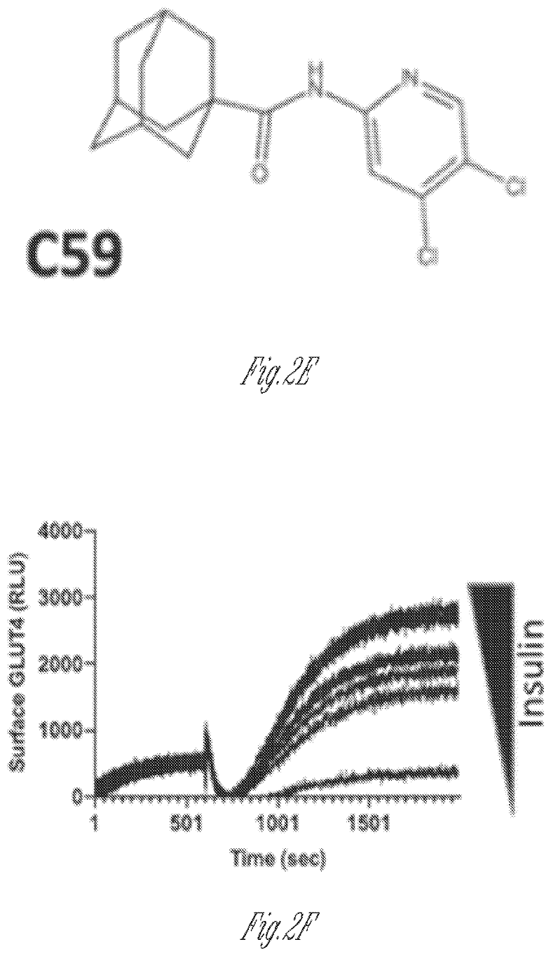

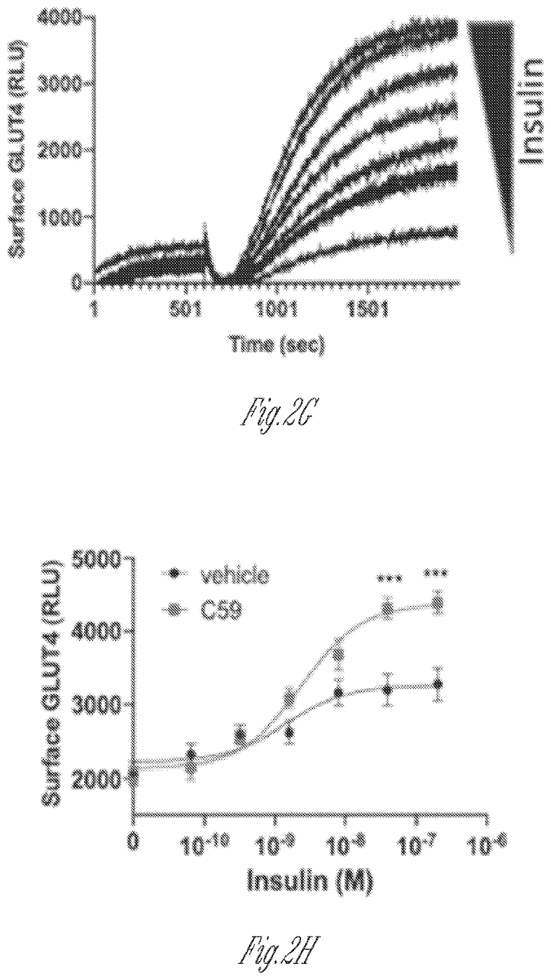

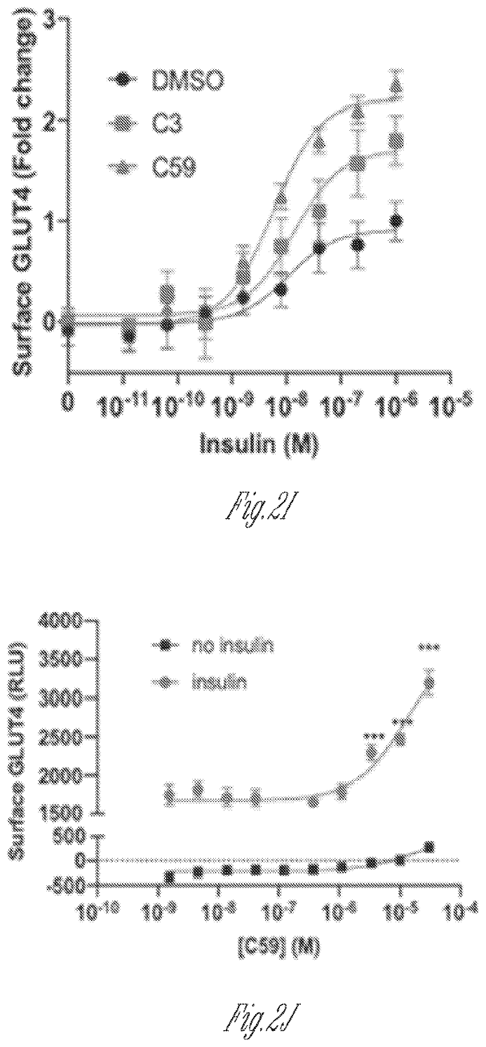

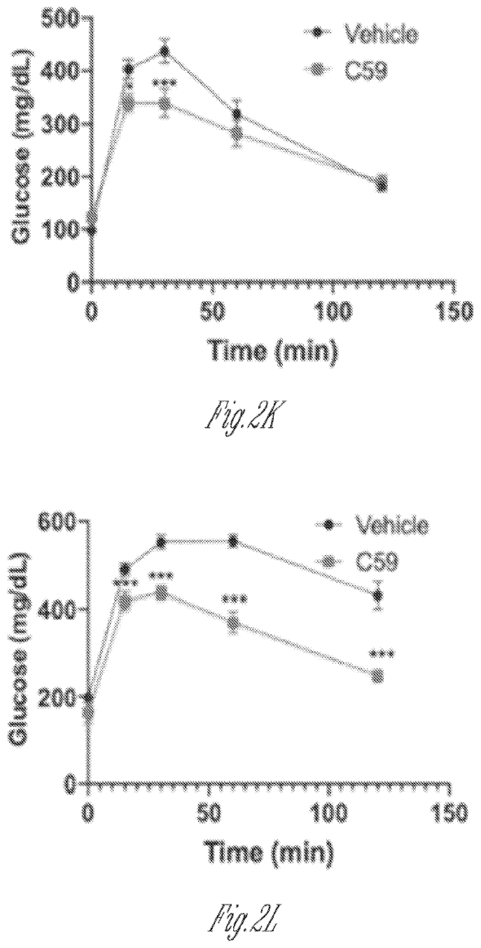

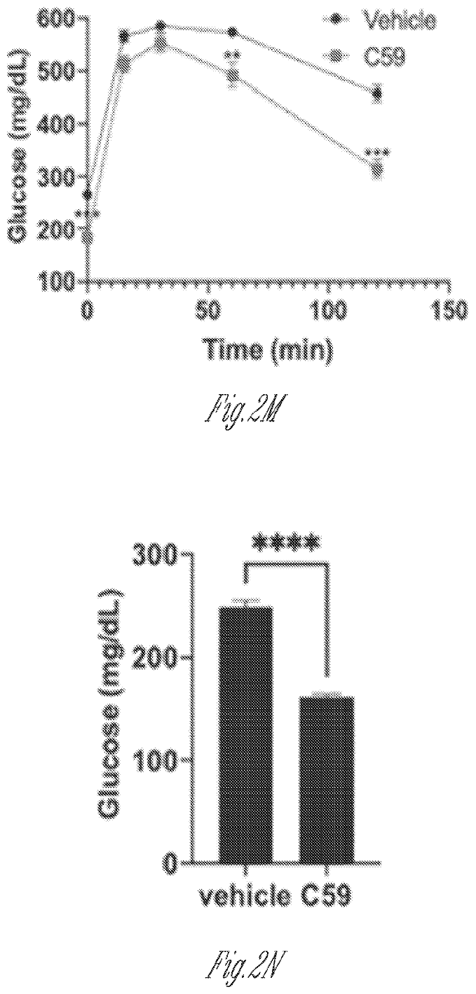

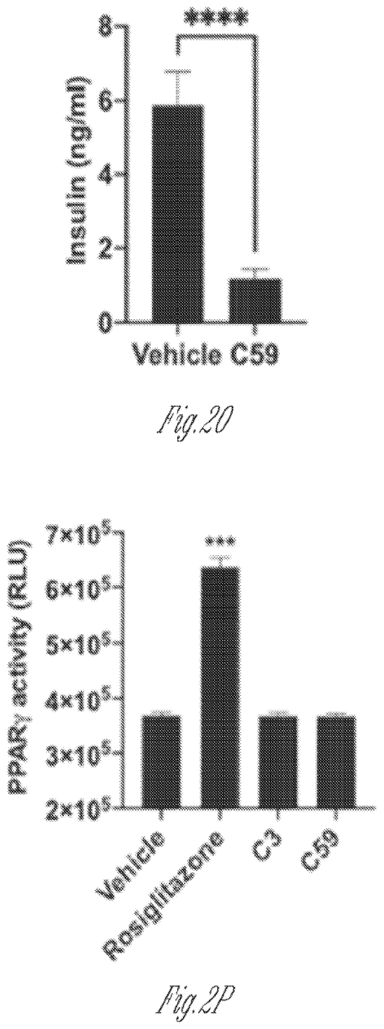

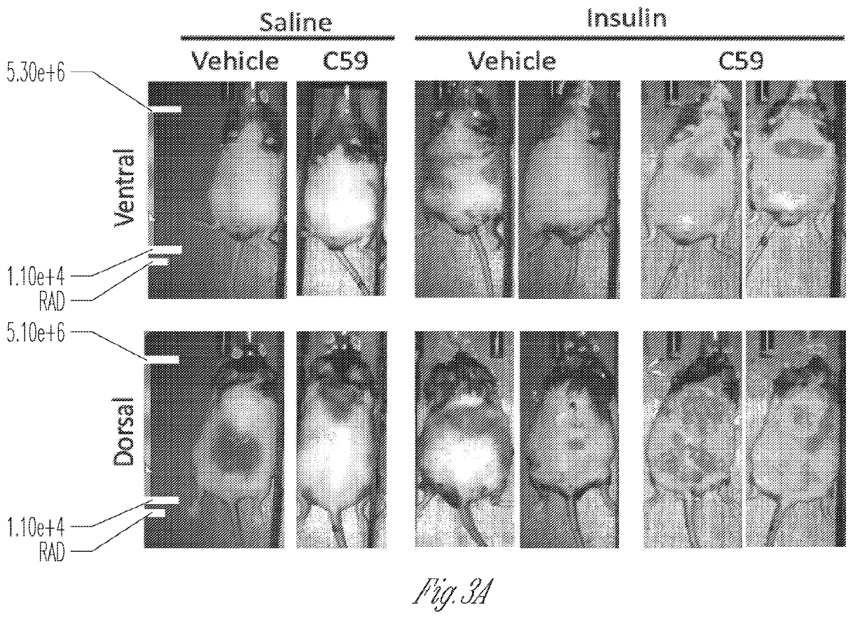

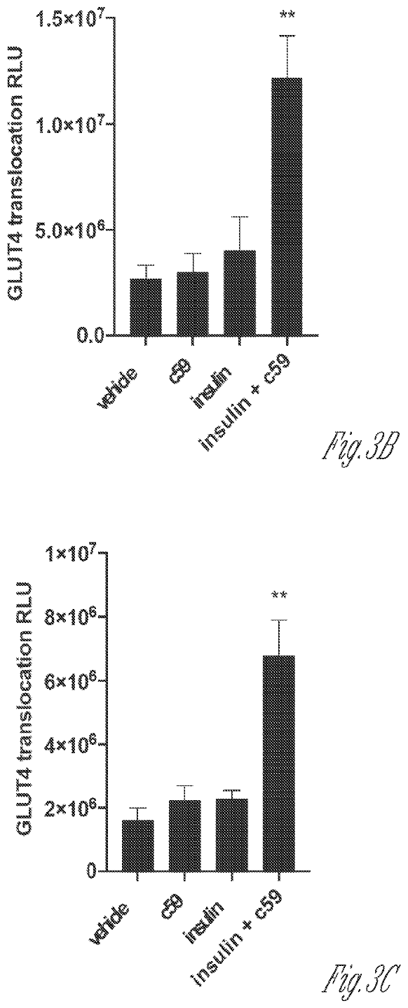

Identification of insulin sensitizers by HTS. Using the described assay, 49,600 compounds from the Chembridge Diverset library were screened in the presence of a submaximal concentration of insulin. Compounds that potentiated the effect of insulin were classified as “hits”. 1264 hits were identified (Table 1), then picked for validation and tested in the absence of insulin to eliminate molecules that promote GLUT4 translocation independently of insulin. 80 validated sensitizers were identified and further characterized pharmacologically. Based on the results, compound C3 which displayed no effect on GLUT4 translocation on its own but significantly potentiated insulin-stimulated GLUT4 translocation in a concentration-dependent manner (FIG. 2B and C) was selected for further study. Consistent with the GLUT4 translocation results, C3 also significantly improved insulin-stimulated glucose uptake in primary adipocytes.

Generating a mouse model for measurement of GLUT4 translocation ex vivo and in-vivo. To enable the validation of identified compounds in a relevant system, a mouse model in which the coding sequence for HiBiT was inserted in the first extracellular loop of endogenous GLUT4 using CRISPR/Cas9 (HBG4 mouse) was prepared. The addition of HiBiT did not alter the tissue distribution of GLUT4 as shown by preparing lysates from multiple tissues and measuring luminescence. GLUT4 was detected in skeletal muscle, white adipose tissue, and brown adipose tissue but not in the brain, spleen, liver, or pancreas, thus, confirming the normal distribution of HiBit-labeled GLUT4 and the ability of this assay to detect endogenous GLUT4 in mouse tissues.

C3 potentiates insulin action in primary adipocytes and in live mice. Using HBG4 mice, insulin stimulated GLUT4 translocation of endogenous GLUT4 was measured in primary skeletal muscle fibers and primary adipocytes. Enhancement of insulin-stimulated translocation of endogenous GLUT4 was also shown in primary adipocytes in the presence of C3. In parallel dishes, increased insulin-stimulated glucose uptake in HBG4 primary adipocyte treated with C3 was found. These results further confirm the insulin sensitizing effect of C3 in a more relevant system than C2C12 myoblast. To determine if the HBG4 mouse can be used to measure GLUT4 translocation in vivo, HBG4 mice were pretreated with daily IP injections of vehicle or 50 mg/Kg C3 for 3 days. On the day of the experiment, mice were injected with vehicle or insulin IP followed by a subcutaneous injection of LgBiT and substrate around the leg muscles. Mice were then anesthetized and placed into an IVIS system to measure luminescence. WT mice were used to measure background and showed little to no signal. Baseline signal was detected in vehicle and C3 treated HBG4 mice. Insulin caused an increase in detected luminescence which was further increased in mice retreated with C3. This result established the HBG4 mouse as the first model in which GLUT4 translocation can be measured in live animals. It also further confirms the insulin-sensitizing activity of C3 in vivo.

C3 improves glucose tolerance and circulating insulin concentration in a model of insulin resistance. To test whether C3 improves glucose handling, WT mice were made insulin-resistant by feeding them a high fat diet (HFD) for a period of 10 weeks. After 10 weeks, mice were injected with vehicle. C3 (20 mg/kg), or the TZD rosiglitazone (20 mg/kg) for 2 weeks while remaining on the HFD. Vehicle treated diet-induced obese (DIO) mice displayed elevated fasting glucose and insulin, hallmarks of insulin resistance. Mice treated with C3 or rosiglitazone both displayed a similar and significantly decrease fasting blood glucose and plasma insulin, thus suggesting an improvement in glycemic control and insulin sensitivity. To further test the in vivo efficacy of C3, a glucose tolerance test (GTT) was perfromed. While vehicle treated DIO mice retained elevated glucose and insulin levels during the duration of the experiment, mice that received C3 or rosiglitazone rapidly cleared glucose and displayed a normal insulin response to the glucose challenge. These results suggest that C3 displays similar in vivo efficacy as the clinically available TZD even before any chemical modification.

C3 is not liver toxic and acts through a different mechanism than TZDs. To determine if C3 treatment caused liver toxicity, plasma levels of liver enzymes ALT and AST were measured. Neither liver toxicity markers were increased in mice treated with C3. DIO mice showed a significant decrease in liver triglyceride content compared to saline treated mice. Deleterious side effect or TZDs are due to their agonism of PPARg. Using a luminescent PPARg reporter assay in vitro, unlike rosiglitazone, C3 had no agonist activity at PPARg and consequently is unlikely to cause the side effects brought upon by TZD treatment.

Generation of derivatives of C3 for improved solubility and composition of matter. One of the carbons of the aromatic ring in C3 was replaced with a nitrogen. This molecule, referred to a C59, like the original C3 compound, enhanced the effect of insulin on GLUT4 translocation. The efficacy of C59 was higher that C3 and the potency of C59 (2.5 μM) was better than C3 by a factor of at least 4. Finally, injection of a single dose of C59 in lean mice resulted in improvement of glucose clearance measured by a glucose tolerance test.

Thus, C59 has insulin sensitizing activity and so is useful as a treatment for type II diabetes.

TABLE 1
HTS Screen results

| | Counts | % |
|---|---|---|
| Library | 49600 | 100 |
| Primary hits | 1264 | 2.5 |
| Validated hits | 692 | 1.4 |
| Insulin dependent hits | 80 | 0.2 |

### EXAMPLE 2

Exemplary variations in the hydrophobic group, linking amide, and aryl/heteroaryl group of C3 are described below.

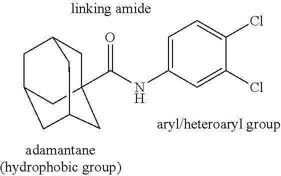 [SMILES: O=C(Nc1ccc(Cl)c(Cl)c1)C12CC3CC(CC(C3)C1)C2]

### Hydrophobic/Adamantane Group:

The adamantane groups may be substituted with a smaller hydrophobic groups like cyclohexyl and benzyl groups in place of adamantane and maintain activity. See examples below:

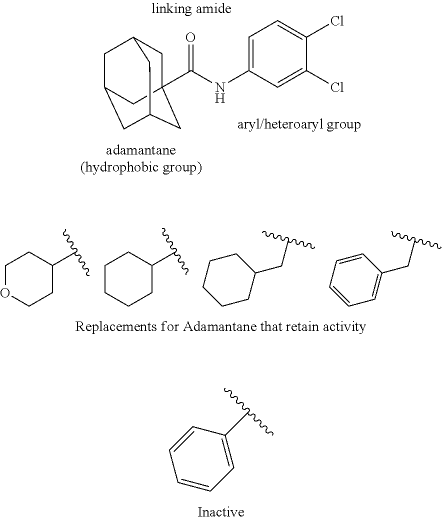

### Aryl/Heteroaryl Group:

Introducing substituted heterocycles in place of the dichloro substituted phenyl ring improved solubility and maintained or improved activity. Exemplary substituted heterocycle substitutions are shown below.

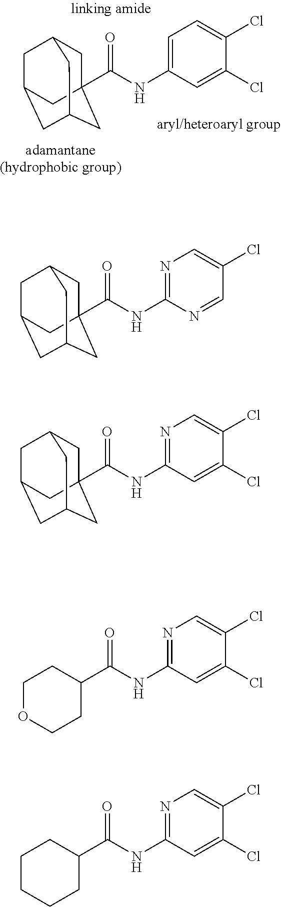 [SMILES: O=C(Nc1ccc(Cl)c(Cl)c1)C12CC3CC(CC(C3)C1)C2 ; O=C(Nc1ncc(Cl)cn1)C12CC3CC(CC(C3)C1)C2 ; O=C(Nc1cc(Cl)c(Cl)cn1)C12CC3CC(CC(C3)C1)C2 ; O=C(Nc1cc(Cl)c(Cl)cn1)C1CCOCC1 ; O=C(Nc1cc(Cl)c(Cl)cn1)C1CCCCC1]

Exemplary compounds similar to those immediately above include:

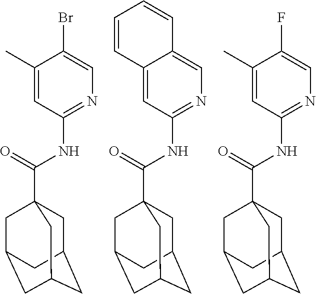

### Amide Linker:

Compounds having alterations in the amide group linking the hydrophobic and aryl/heteroaryl parts, even without changes to the adamantane and aryl/heteroaryl parts of the molecule, are likely to have the activity of C59.

A representative example is shown below.

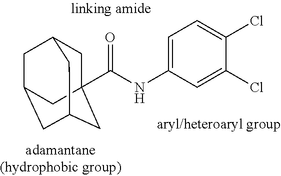 [SMILES: O=C(Nc1ccc(Cl)c(Cl)c1)C12CC3CC(CC(C3)C1)C2]

### EXAMPLE 3

### Development of an HIS Assay for Real-Time Measurement of GLUT4 Translocation

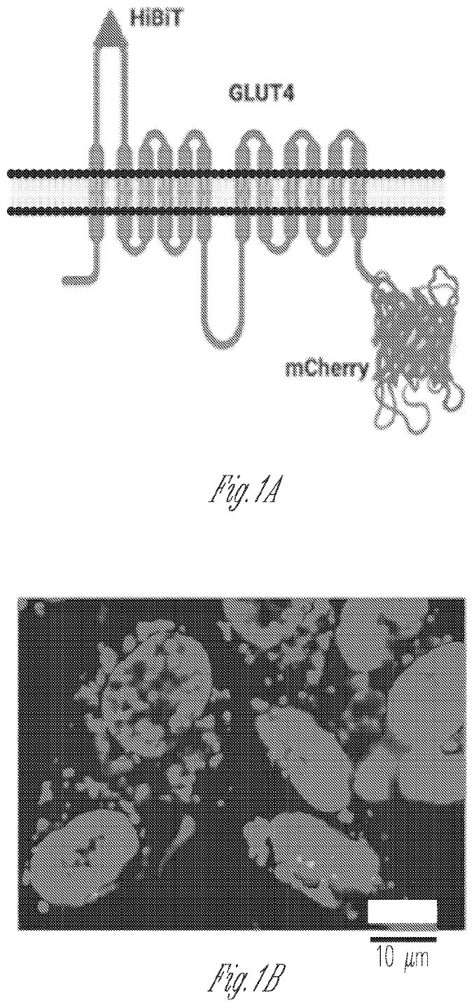

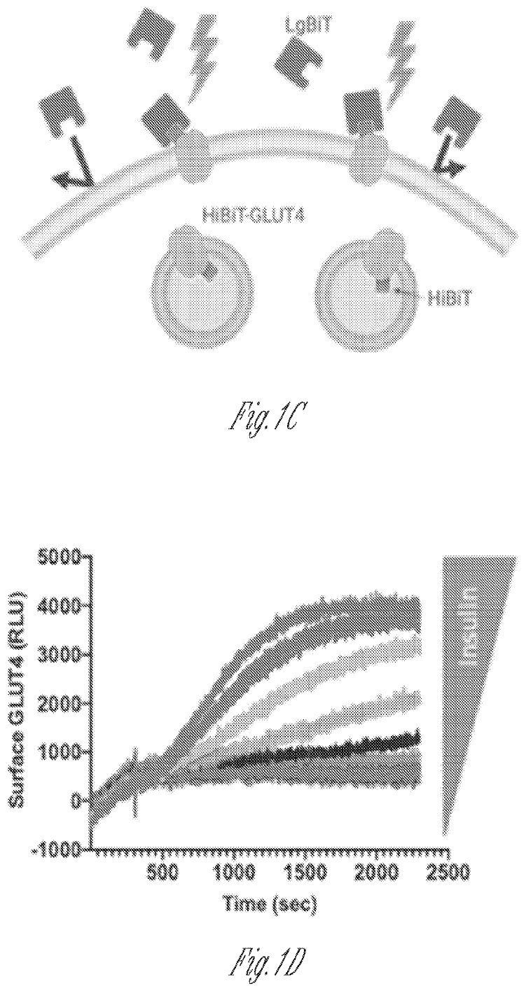

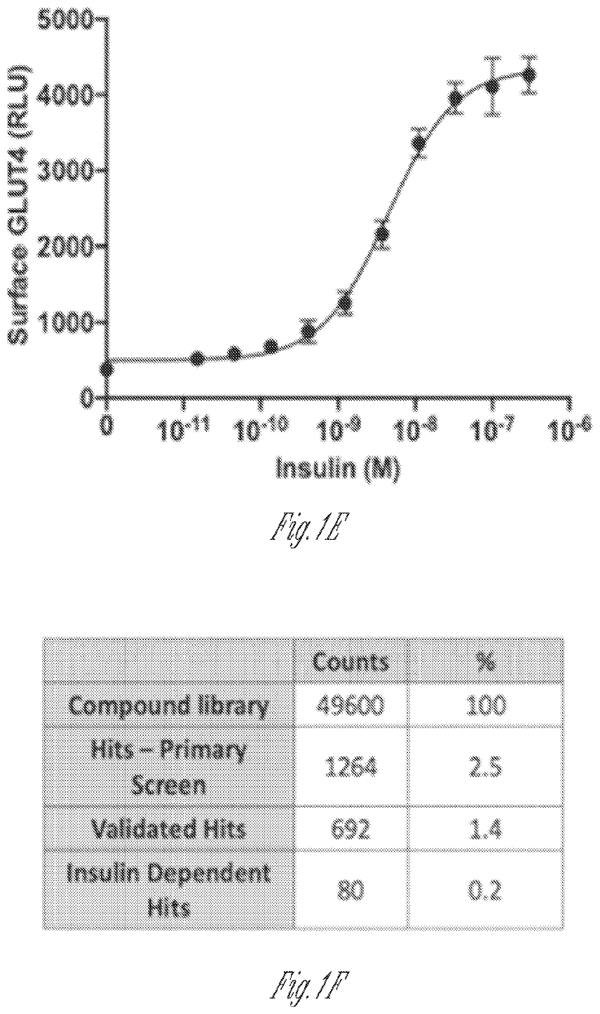

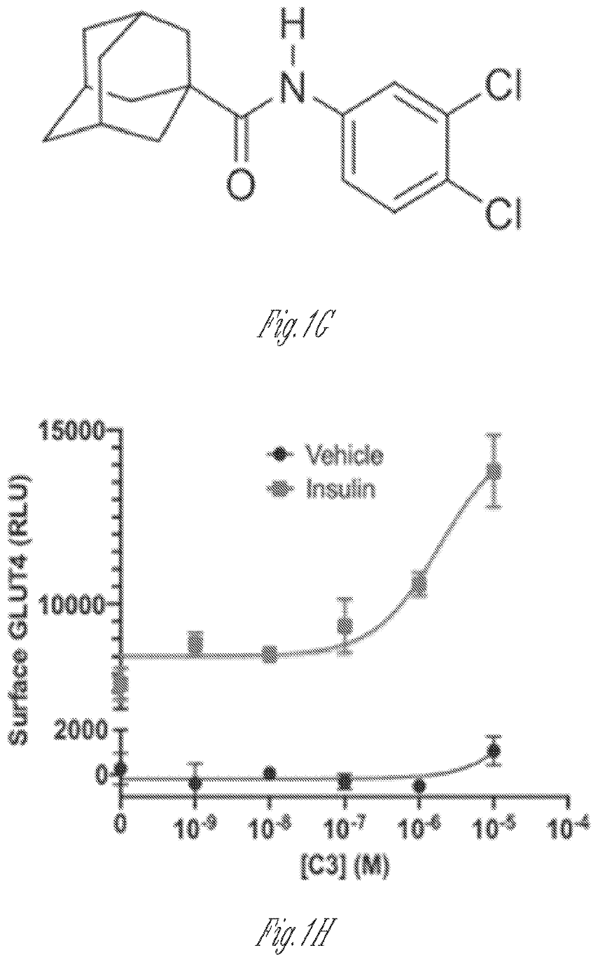

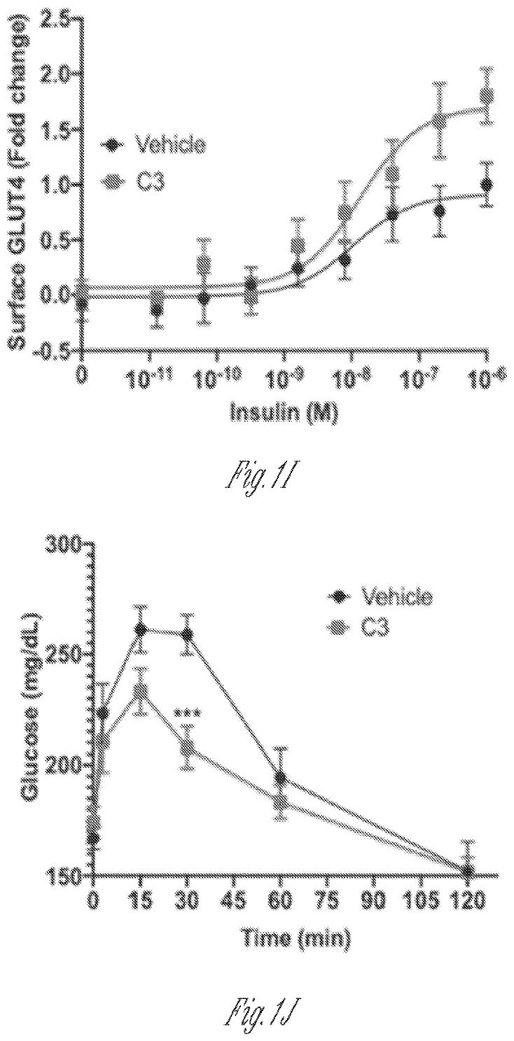

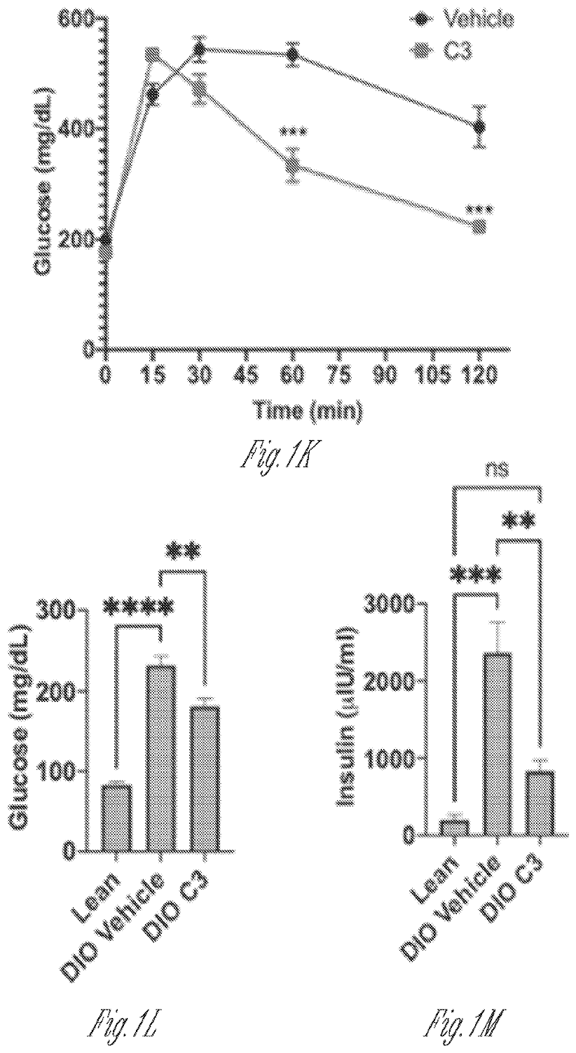

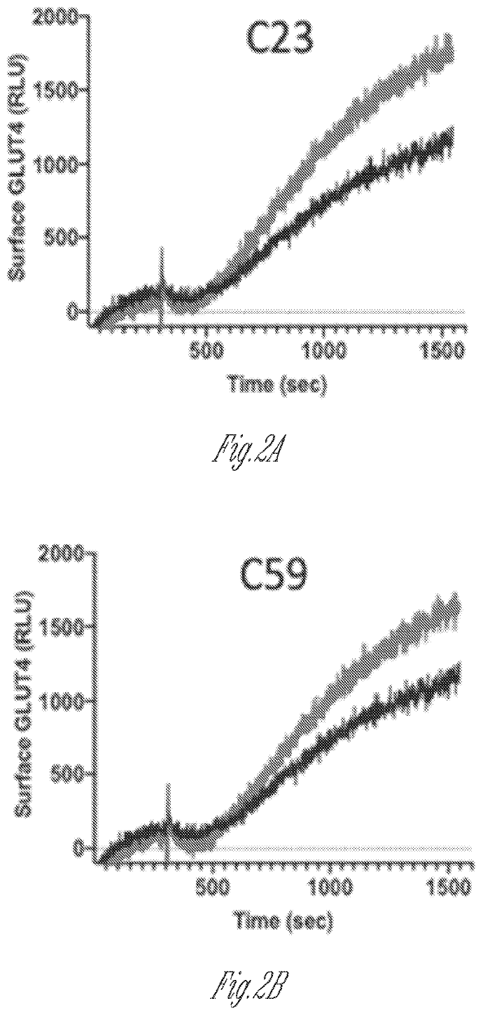

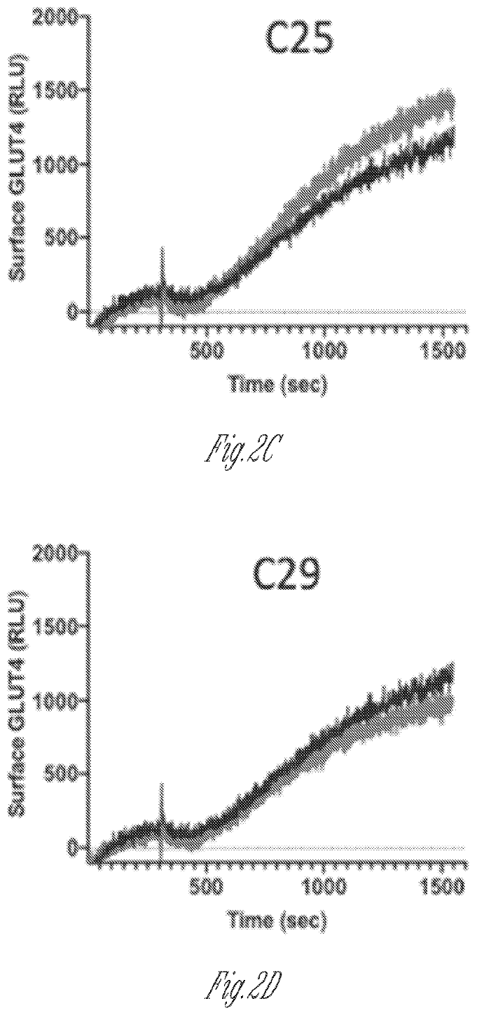

An assay to measure GLUT4 translocation in a homogenous and HIS amenable manner using NanoBit technology was developed. This technology relies on the rapid recombination of 2 fragments of the Nanoiuc luciferase (HiBit and LgBiT) with high affinity for each other. Fragments have no enzymatic activity on their own but generate a bright luminescent signal when recombined. A C2C12 myoblast cell line stably expressing HiBiT-GLUT4 (HBG4), in which the small 11 amino acid HiBiT fragment was inserted into the first extracellular loop of GLUT4 was prepared. A C-terminal mCherry was also added for microscopic detection (FIGS. 1A and B). The C2C12 line was chosen as an in-vitro model because it is derived from skeletal myoblasts and translocates GLUT4 in response to insulin. Detection of plasma membrane density of HBG4 is achieved by adding recombinant LgBit and substrate to live cells (FIG. 1C). The assay can be conducted as a kinetic assay by adding LgBiT and substrate at the beginning of the experiment and monitoring real-time insulin-stimulated GLUT4 translocation (FIGS. 1D and E).

Identification of insulin sensitizers by HTS. Using the described assay, 50,000 compounds from the Chembridge Diverset library were screened in the presence of a submaximal concentration of insulin. Compounds that potentiated the effect of insulin were classified as “hits”. 1264 hits were identified (FIG. 1F), cherry picked for validation and tested in the absence of insulin to eliminate molecules that promote GLUT4 translocation independently of insulin. 80 validated sensitizers were identified (FIG. 1F) and further characterized pharmacologically. Based on the results, compound C3 (FIG. 1G), which displayed no effect on GLUT4 translocation on its own but significantly potentiated insulin-stimulated GLUT4 translocation in a concentration-dependent manner (FIGS. 1H and I) was further characterized.

C3 improves glucose tolerance in lean and obese insulin resistant mice. To test whether C3 improves glucose handling, lean mice were injected with vehicle or C3 30 minutes before performing a glucose tolerance test. C3 significantly accelerated glucose clearance in lean mice (FIG. 1J). To further determine if C3 can reverse glucose intolerance, WT mice were made insulin-resistant by feeding them a high fat diet (HFD) for a period of 10 weeks. Diet induced obese (DIO) mice were then injected with vehicle or C3 for 2 weeks while remaining on HFD. GTT showed a very significant improvement in glucose handling in DIO mice treated with C3 compared to control (FIG. 1M). Additionally, vehicle treated DIO mice displayed elevated fasting glucose and insulin, hallmarks of insulin resistance. C3 significantly decreased fasting blood glucose (FIG. 1L) and plasma insulin (FIG. 1M), thus further demonstrating an improvement in glycemic control and insulin sensitivity. Together those results establish in vivo efficacy of C3 for the treatment of insulin resistance.

Structure activity relationship studies for C3. Several derivatives of C3 were prepared to identify important groups and improve solubility and efficacy. A few examples of compounds that retained activity, C23 and C59 (FIGS. 2A and B) and a derivative with no activity C29 (FIG. 2C) are shown as example. C59 displayed improved solubility in aqueous solution, improved potency (FIG. 2D) and improved efficacy (FIGS. 2E-G). Importantly, unlike rosiglitazone (TZD approved for clinical use), neither C3 nor C59 have PPARγ agonist activity. Like C3, C59 treatment drastically improves glucose handling in DIO mice (FIG. 2I).

Generating a mouse model to measure GLUT4 translocation in-vivo. To enable the validation of identified compounds in a relevant system, a mouse model was developed in which the coding sequence for HiBiT was inserted in the first extracellular loop of endogenous GLUT4 using CRISPR/Cas9 (HBG4 mouse). The addition of HiBiT did not alter the tissue distribution of GLUT4 as determined by preparing lysates from multiple tissues and measuring luminescence. GLUT4 was detected in skeletal muscle, white adipose tissue, and brown adipose tissue but not in the brain, spleen, liver, or pancreas, thus confirming the normal distribution of HiBit-labeled GLUT4 and the ability of this assay to detect endogenous GLUT4 in mouse tissues.

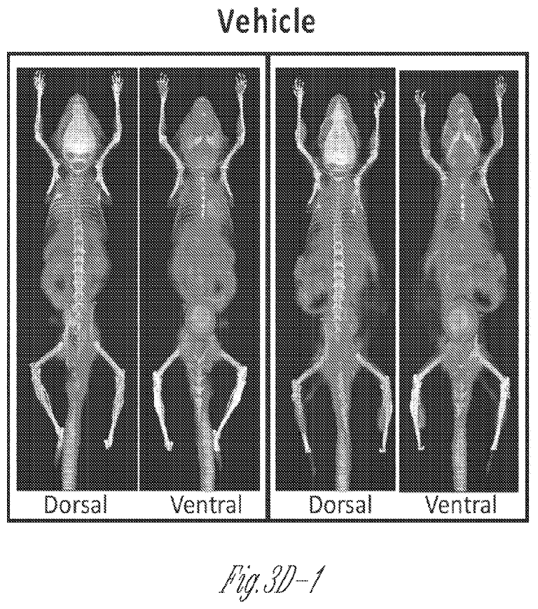

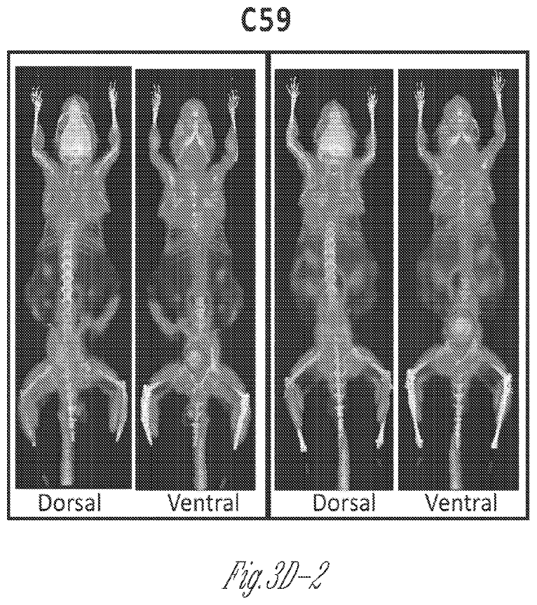

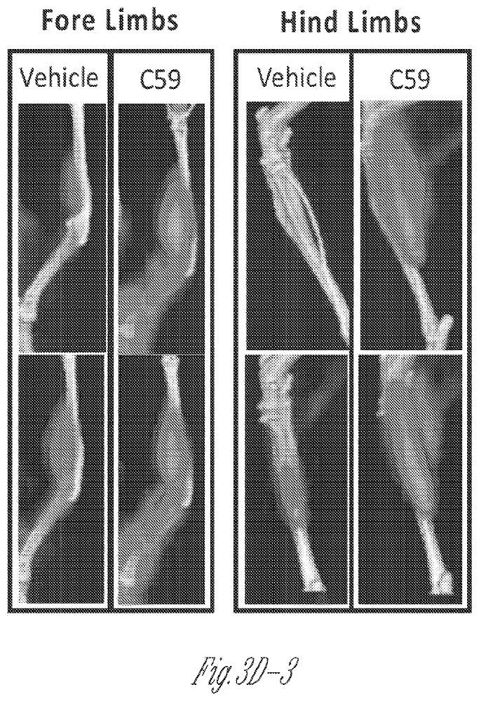

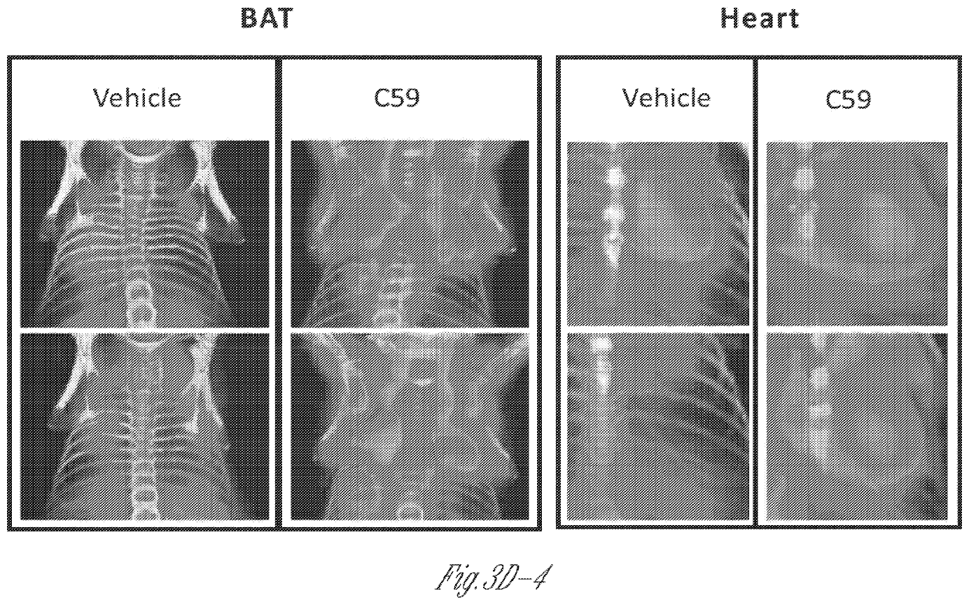

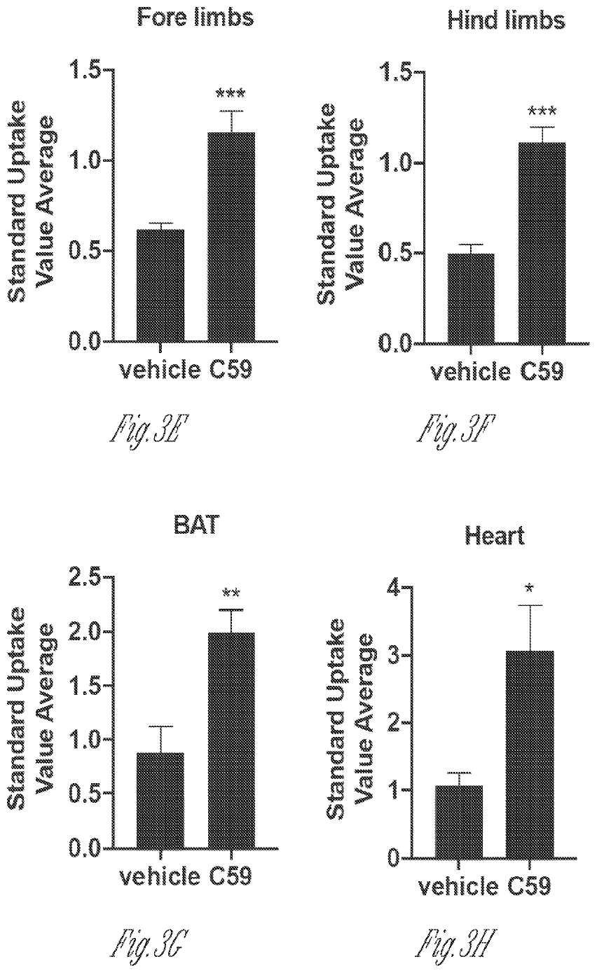

DIO HBG4 mice were treated with vehicle or C59 for 2 weeks and injected i.v. with an adenovirus coding for a secreted form of LgBiT (allowing LgBiT production and secretion predominantly by the liver into the circulation) 2 days prior to the experiment. Animal were then injected with saline or insulin 10 minutes before i.v. injection of substrate and luminescence imaging using an IVIS system. C59 treatment very significantly improved insulin-stimulated GLUT4 translocation (FIGS. 3A-C) thus confirming in-vivo the activity of C59 identified during the HTS assay on cells.

C59 improves glucose uptake in storage tissues of DIO mice. Since insulin resistance results in a failure to transport glucose from the circulation to tissues that can use it as a source of energy or store it, it was tested whether treating insulin resistant DIO mice with C59 results in an increase in glucose uptake in GLUT4 expressing tissues. For this, insulin resistant DIO mice (fed a HFD for 10 weeks) were fed HFD or HFD supplemented with C59 for two weeks before injection of ¹⁸F-FDG and uptake of the radioactive glucose tracer was measured by PET/CT.

scan. In agreement with the data demonstrating increased GLUT4 translocation in C59 treated mice, C59 significantly increased ¹⁸F-FDG uptake in skeletal muscles, WAT, BAT and heart tissues (FIGS. 3D-H).

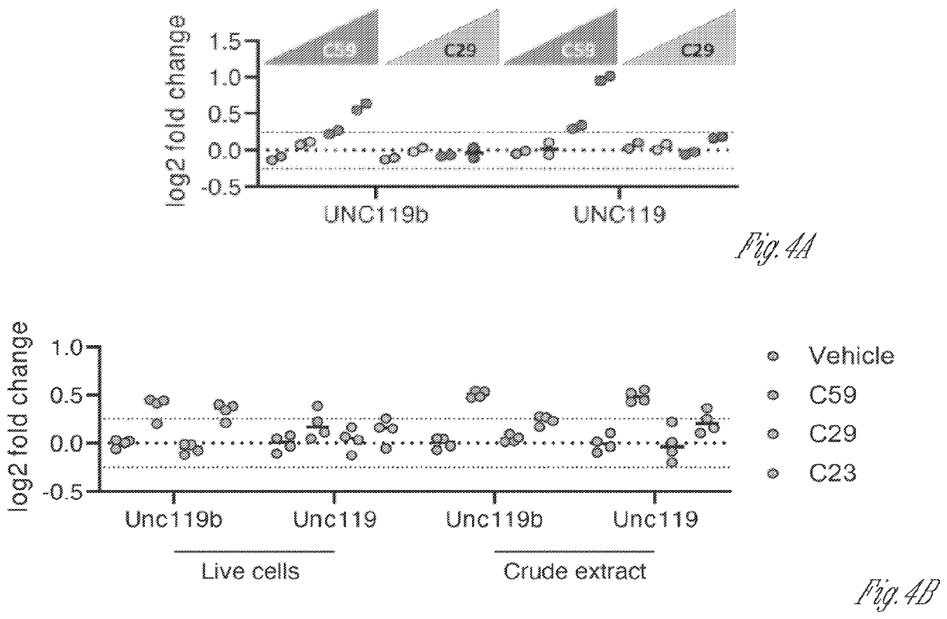

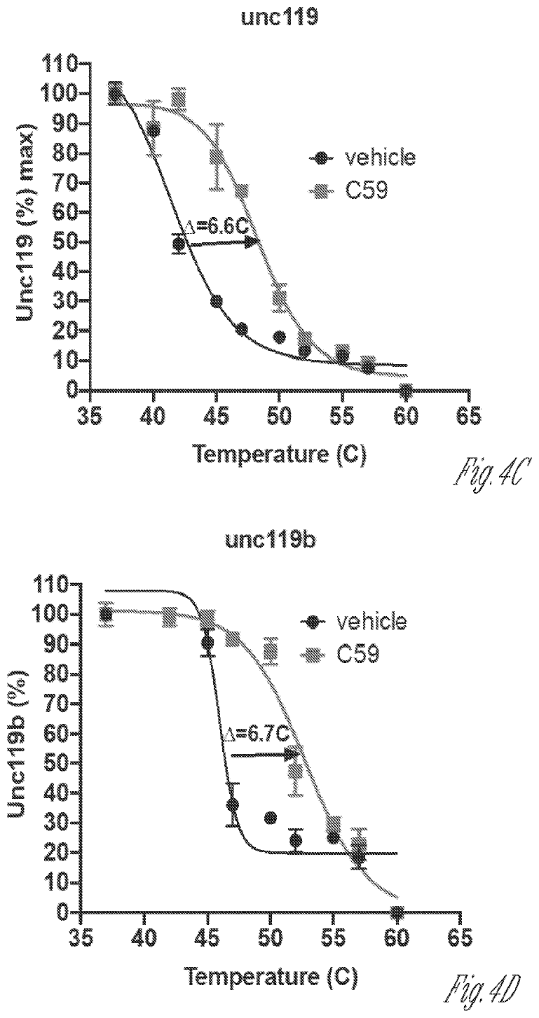

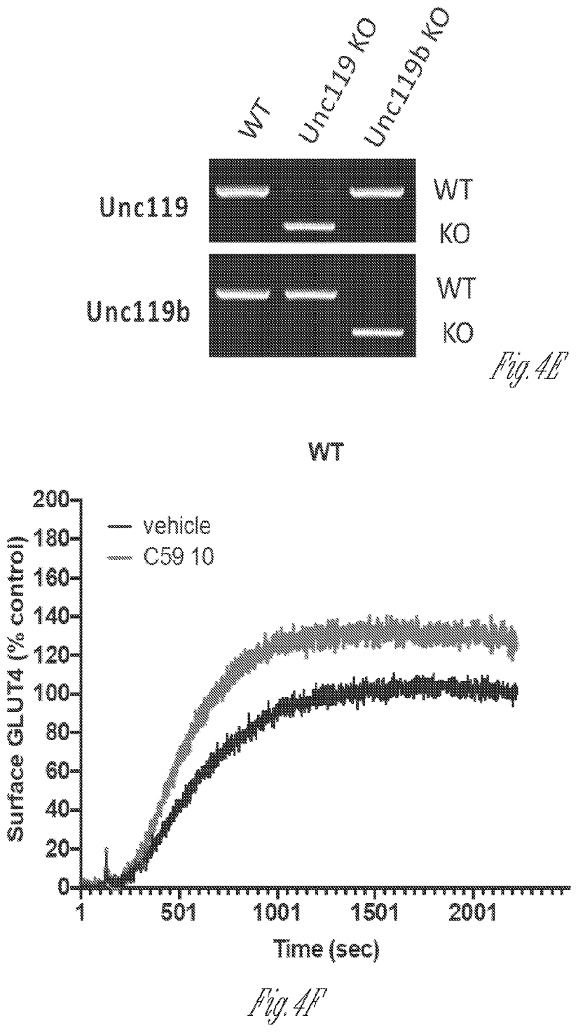

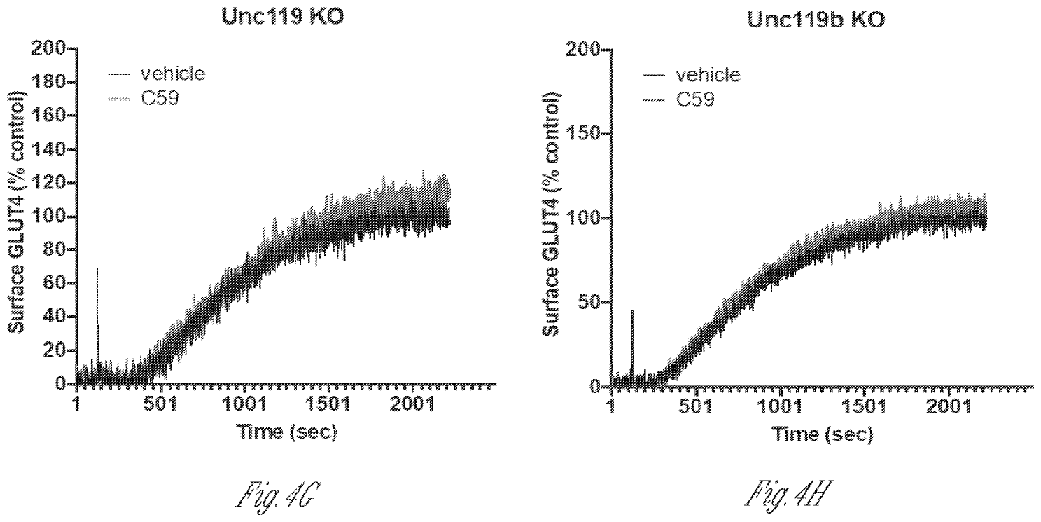

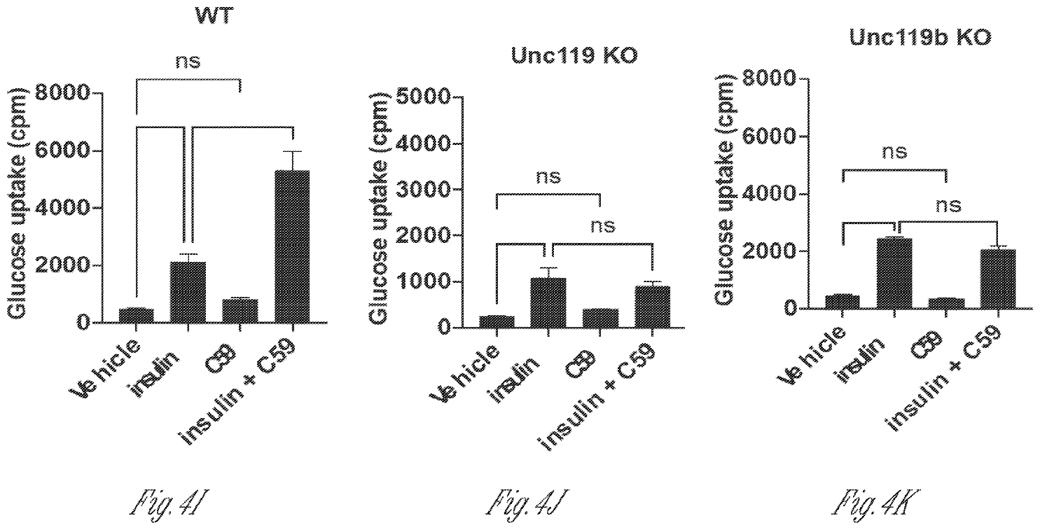

Identification of the molecular target of C59. Since the discovery of C59 as an insulin sensitizer was achieved using a phenotypic screen, the molecular target of the compound was unknown. To identify the target, a Proteome Integral Solubility Alteration (PISA) assay was used. Binding of small molecules to proteins can alter their thermostability and changes in thermostability can be measured by heating either live cells or cell lysates incubated with compounds to different temperatures, removing thermally destabilized and precipitated proteins by centrifugation and quantitatively identifying enrichment of specific proteins using TMT labeling and mass spectrometry, Live cells were incubated with either 0 (DMSO), 5 mM, 25 mM or 125 mM of C59 or C29 (inactive derivative of compound) before processing for PISA. we found that C59 increased the thermostability of Unc119 and Unc119b in a concentration dependent manner compared to controls (DMSO and C29) (FIG. 4A). To further demonstrate a direct interaction of C59 with Unc119 and Unc119b, PISA was performed with 2 active compounds (C59 and C23) and 2 controls (DMSO and C29) in lysates (crude extract). Here again it was found that both C59 and C23 increased the thermostability of Unc119 and Unc119b but C29 did not (FIG. 4B). To measure the shift in thermal stability, constructs coding for Unc119-HiBiT and Unc119b-HiBit were generated, thus allowing the quantitative detection of those proteins by adding LgBiT and furimazin to the lysates and measuring luminescence. Crude extract from cells transfected with Unc119-HiBiT or Unc119b-HiBiT, were incubated with DMSO or C59 for 10 minutes, aliquoted, heated for 3 minutes at the indicated temperature and allowed to cool down to room temperature before adding LgBiT and furimazin and reading luminescence on a plate reader. C59 caused a 6.6 C. and 6.7 C. thermostability shift for Unc119 and Unc119b respectively (FIGS. 4C and D), thus further demonstrating binding of C59 to those proteins.

Unc119 and Unc119b are important for the insulin sensitizing effect of C59. After identifying Unc119 proteins as molecular targets for C59, it was tested whether those proteins were responsible for the insulin sensitizing activity of C59. To this end Unc119 and Unc119b KO HBG4 cells were generated using CRISPR/Cas9 technology. For each of those cell lines two gRNA surrounding the start codon were used, thus resulting in a deletion of a fragment of the Unc119 genes that includes the ATG. Deletion of Unc119 and Unc119b in clonal cell lines were screened and verified by PCR (FIG. 4E). The effect of C59 on insulin-stimulated. GLUT4 translocation in WT (FIG. 9F), Unc119 KO (FIG. 9G) and Unc119b KO (FIG. 9H) cells was measured, Whereas C59 increased GLUT4 translocation in WT cells as has shown before, C59 failed to enhance insulin action in Unc119 KO and Unc119b KO cell lines, thus suggesting that those proteins are the C59 targets responsible for the insulin sensitizing action of C59. To further test this finding, 3H-Deoxy-D-Glucose uptake in WT, Unc119 KO and Unc119b KO HBG4 cells treated with vehicle, insulin, C59 alone and C59 with insulin was measured. As expected, insulin increased glucose uptake in all 3 cell lines and C59 alone did not stimulate glucose uptake (FIGS. 9I-K). However, while C59 significantly increased insulin stimulated glucose uptake in WT cells (FIG. 9I), it failed to do so in Unc119 KO (FIG. 4J) and Unc119b KO (FIG. 4K) cells. This result confirms that C59 action on Unc119 proteins is important for sensitizing insulin activity.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method of preventing, inhibiting or treating a mammal having type II diabetes, comprising administering an effective amount of a compound having the structure (I): 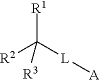
wherein
each of R1, R2, and R3 is independently H, alkyl, aryl, or heteroaryl, or two or three of R1, R2, and R3 taken together with the carbon to which they attach form a cycloalkyl ring;
L is a linker selected from amide, oxime, carbamate, carbamide, triazole, oxazole, oxadiazole, or sulfonamide; and
A is aryl or heteroaryl.

2. The method of claim 1, wherein A is substituted aryl, substituted heteroaryl, or unsubstituted heteroaryl, is substituted with one, two, or three chloro, is a heteroaryl, is pyridine, pyrimidine, pyridazine, pyrazine, quinoline, isoquinoline, or naphthylene, is a dichlorophenvl, optionally substituted with one or more additional substituent, is other than phenyl, is 2-pyridinyl, 2-pyrimidinyl, or 3-isoquinolyl, is a 6-membered ring has a halogen para to the linkage to L, is a 6-membered ring having a 4-halo, 5-halo, or both, is a 6-membered ring having a 4-chloro, 5-chloro, or both, or is a 6-membered heteroaryl having a 4-chloro, 5-chloro, or both.

3-12. (canceled)

13. The method of claim 1 wherein the compound has structure (II): 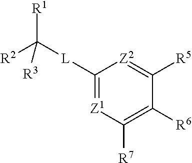
wherein
each of R¹, R², and R³ is independently H, alkyl, aryl, or heteroaryl, or two or three of R1, R², and R³ taken together with the carbon to which they attach form a cycloalkyl ring;
L is a linker selected from amide, oxime, carbamate, sulfonamide, or carbamide;
each of Z¹ and Z² is independently CR⁸, CR⁹, or N;
each of R⁵, R⁶, R⁷, R⁸, and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring, and
when Z¹ and Z² are other than N, then at least one of R⁵, R⁶, R⁷, R⁸, and R⁹ is other than hydrogen.

14. The method of claim 1 wherein the compound has structure (III): 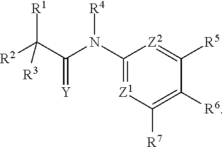
wherein
each of R¹, R², and R3 is independently H, alkyl, aryl, or heteroaryl, or two or three of R1, R², and R³ taken together with the carbon to which they attach form a cycloalkyl ring;
L is a linker selected from amide, oxime, carbamate, sulfonamide, or carbamide,
Y is O, S, or NR10;
each of R4 and R10 is independently hydrogen, alkyl, aryl, hydroxyl;
each of Z¹ and Z² is independently CR⁸, CR⁹, or N;
each of R⁵, R⁶, R⁷, R⁸, and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they attach form an aryl or heteroaryl ring, and
when Z¹ and Z² are other than N, then at least one two of R⁵, R⁶, R⁷, R⁸, and R⁹ is other than hydrogen.

15. A compound having the structure (II): 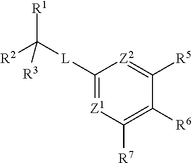
wherein
each of R¹, R², and R³ is independently H, alkyl, aryl, or heteroaryl, or two or three of R1, R², and R³ taken together with the carbon to which they attach form a cycloalkyl ring;
L is a linker selected from amide, oxime, carbamate, carbamide, triazole, oxazole, oxadiazole, or sulfonamide;
each of Z¹ and Z² is independently CR⁸, CR⁹, or N;
each of R⁵, R⁶, R⁷, R⁸ and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sultonate, or phosphonate, or two of R⁵, R⁶, R⁷, R⁸, and R⁹ taken together with the carbons to which they attach form an aryl or heteroaryl ring; and
when Z¹ and Z² are CR⁸ or CR⁹, or when R¹, R², and R³ taken together are adamantyl, then at least one two of R⁵, R⁶, R⁷, R⁸, and R⁹ is other than hydrogen, and at least one of R⁵, R⁶, R⁷, R⁸, and R⁹ other than fluoro, chloro, bromo, and iodo.

16. The compound of claim 15 haying structure (III): 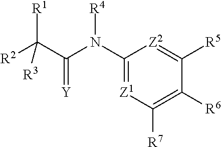
wherein
each of R¹, R², and R3 is independently H, alkyl, aryl, or heteroaryl, or two or three of R1, R², and R³ taken together with the carbon to which they attach form a cycloalkyl ring;
L is a linker selected from amide, oxime, carbamate, sulfonamide, or carbamide;
Y is O, S, or NR10;
each of R4 and R10 is independently hydrogen, alkyl, aryl, hydroxyl;
each of Z¹ and Z² is independently CR⁸, CR⁹, or N;
each of R⁵, R⁶, R⁷, R⁸, and R⁹, if present, is independently H, fluoro, chloro, bromo, iodo, amino, amido, alkyl, alkoxy, alkylamino, alkylthio, alkylamido, formyl, acyl, alkoxycarbonyl, acyloxy, aryloxy, arylamino, arylthio, arylcarbonyl, arylamido, aryloxycarbonyl, hydroxy, amino, cyano, nitro, azido, thio, alkylsulfonyl, alkylsulfinyl, carbonate, sulfonate, or phosphonate, or two of R⁵, R⁶, R⁷, R⁸, and R⁹ taken together with the carbons to which they, attach form an aryl or heteroaryl ring; and
when Z¹ and Z² are CR⁸ or CR⁹, or when R¹, R², and R3 taken together are adamantyl, then at least one two of R⁵, R⁶, R⁷, R⁸, and R⁹ is other than hydrogen, and at least one of R⁵, R⁶, R⁷, R⁸, and R⁹ other than fluoro, chloro, bromo, and iodo.

17. The method of claim 14, wherein Y is O, or N—OH;

18. The method of claim 1, wherein three of R¹, R², and R³ taken together with the carbon to which they attach form a cycloalkyl, wherein R¹, R², and R³ taken together with the carbon to which they attach form an adamantyl ring, wherein two of R¹, R², and R³ taken together with the carbon to which they attach form a cycloalkyl or wherein two of R¹, R², and R3 taken together with the carbon to which they attach form a cyclohexane or tetrahydropyran.

19-21. (canceled)

22. The method of claim 1, wherein at least one of Z¹ and Z² is N, wherein Z¹ and Z² are independently CR⁸ or CR⁹ and at least two of R⁵, R⁶, R⁷, R⁸, and R⁹ are chloro and at least one of R⁵, R⁶, R⁷, R⁸, and R⁹ are other than chloro and other than hydrogen, wherein at least two of R⁵, R⁶, R⁷, R⁸, and R⁹ is chloro fluoro, bromo or iodo.

23-24. (canceled)

25. The method of claim 1, wherein R⁶ is chloro, fluoro, bromo, or iodo.

26. The method of claim 1, wherein one of R⁵ and R⁷ is methyl, chloro, flouoro, or bromo.

27. The method of claim 1, wherein the compound has the structure:

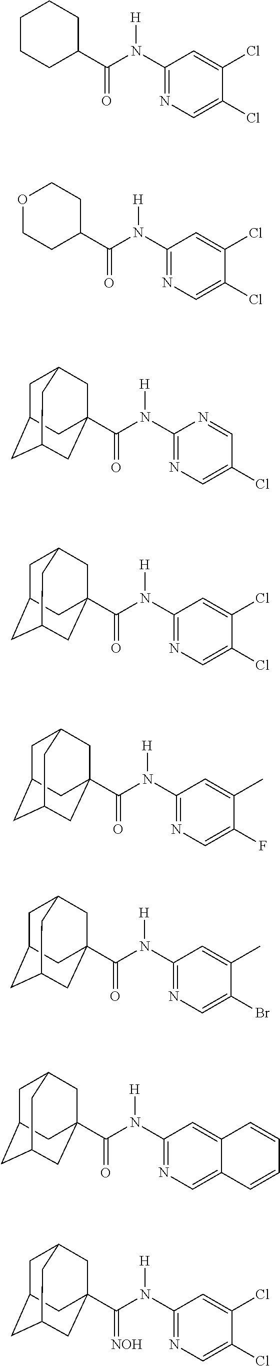

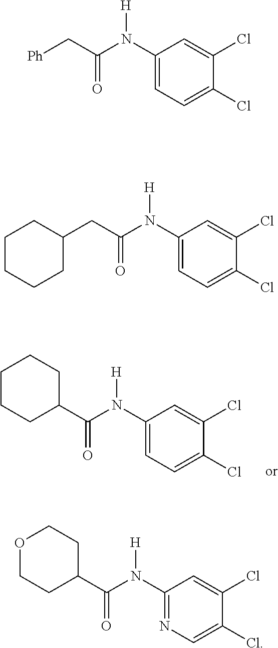 [SMILES: O=C(Cc1ccccc1)Nc1ccc(Cl)c(Cl)c1 ; O=C(CC1CCCCC1)Nc1ccc(Cl)c(Cl)c1 ; O=C(Nc1ccc(Cl)c(Cl)c1)C1CCCCC1 ; O=C(Nc1cc(Cl)c(Cl)cn1)C1CCOCC1]

28. The method of claim 1, wherein the compound has the structure:

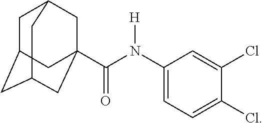 [SMILES: O=C(Nc1ccc(Cl)c(Cl)c1)C12CC3CC(CC(C3)C1)C2]

29. The method of claim 1 wherein the mammal is a human.

30. The method of claim 1 wherein the compound is systemically administered.

31. The method of claim 1 wherein the compound is orally administered.

32. The method of claim 1 wherein the composition is a sustained release dosage form.

33. (canceled)

40. A method of using the non-human recombinant mammal of claim 45 or isolated mammalian cells comprising an expression cassette encoding a fusion polypeptide having at least 80% amino acid sequence identity to GLUT4 sequences in SEQ ID NO:1, 10 or 11, wherein the fusion polypeptide comprises at least one tag to screen for insulin sensitizers, comprising: contacting one or more test compounds with the non-human recombinant mammal or the isolated mammalian cells in the presence of insulin, and detecting or determining whether the polypeptide has enhanced translocation to the plasma membrane in the recombinant mammal or the isolated mammalian cells relative to a corresponding non-human recombinant mammal or corresponding mammalian cells contacted with the one or more test compounds in the absence of insulin.

41-44. (canceled)

45. A non-human recombinant mammal expressing a fusion polypeptide having at least 80% amino acid sequence identity to GLUT4 sequences in SEQ ID NO:1, 10 or 11, wherein the fusion polypeptide comprises at least one tag.

46-47. (canceled)


SEQUENCE LISTING (22 sequences)

SEQ ID NO: 1 - protein, 509 aa, Homo sapiens
MPSGFQQIGSEDGEPPQQRVTGTLVLAVFSAVLGSLQFGYNIGVINAPQKVIEQSYNETWLGRQGPEGPSSIPPGTLTTLWALSVAIFSVGGMISSFLIGIISQWLGRKRAMLVNNVLAVLGGSLMGLANAAASYEMLILGRFLIGAYSGLTSGLVPMYVGEIAPTHLRGALGTLNQLAIVIGILIAQVLGLESLLGTASLWPLLLGLTVLPALLQLVLLPFCPESPRYLYIIQNLEGPARKSLKRLTGWADVSGVLAELKDEKRKLERERPLSLLQLLGSRTHRQPLIIAVVLQLSQQLSGINAVFYYSTSIFETAGVGQPAYATIGAGVVNTVFTLVSVLLVERAGRRTLHLLGLAGMCGCAILMTVALLLLERVPAMSYVSIVAIFGFVAFFEIGPGPIPWFIVAELFSQGPRPAAMAVAGFSNWTSNFIIGMGFQYVAEAMGPYVFLLFAVLLLGFFIFTFLRVPETRGRTFDQISAAFHRTPSLLEQEVKPSTELEYLGPDEND

SEQ ID NO: 2-9 - skipped (8 entries)

SEQ ID NO: 10 - protein, 802 aa, Artificial Sequence
  A synthetic sequence
MPSGFQQIGSDDGEPPRQRVTGTLVLAVFSAVLGSLQFGYNIGVINAPQKVIEQSYNATWLGRQGPGRASTPVSGGSVSGWRLFKKISGSSGGSSGREAEEQKLISEEDLLKRVAGGPDSIPQGTLTTLWALSVAIFSVGGMISSFLIGIISQWLGRKRAMLANNVLAVLGGALMGLANAAASYEILILGRFLIGAYSGLTSGLVPMYVGEIAPTHLRGALGTLNQLAIVIGILVAQVLGLESMLGTATLWPLLLALTVLPALLQLILLPFCPESPRYLYIIRNLEGPARKSLKRLTGWADVSDALAELKDEKRKLERERPMSLLQLLGSRTHRQPLIIAVVLQLSQQLSGINAVFYYSTSIFESAGVGQPAYATIGAGVVNTVFTLVSVLLVERAGRRTLHLLGLAGMCGCAILMTVALLLLERVPAMSYVSIVAIFGFVAFFEIGPGPIPWFIVAELFSQGPRPAAMAVAGFSNWTCNFIVGMGFQYVADAMGPYVFLLFAVLLLGFFIFTFLKVPETRGRTFDQISAAFRRTPSLLEQEVKPSTELEYLGPDENDGDPPEFTTMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 11 - protein, 536 aa, Artificial Sequence
  A synthetic sequence
MPSGFQQIGSDDGEPPRQRVTGTLVLAVFSAVLGSLQFGYNIGVINAPQKVIEQSYNATWLGRQGPGSSGGSSGVSGWRLFKKISGSSGGSSGGGPDSIPQGTLTTLWALSVAIFSVGGMISSFLIGIISQWLGRKRAMLANNVLAVLGGALMGLANAAASYEILILGRFLIGAYSGLTSGLVPMYVGEIAPTHLRGALGTLNQLAIVIGILVAQVLGLESMLGTATLWPLLLALTVLPALLQLILLPFCPESPRYLYIIRNLEGPARKSLKRLTGWADVSDALAELKDEKRKLERERPMSLLQLLGSRTHRQPLIIAVVLQLSQQLSGINAVFYYSTSIFESAGVGQPAYATIGAGVVNTVFTLVSVLLVERAGRRTLHLLGLAGMCGCAILMTVALLLLERVPAMSYVSIVAIFGFVAFFEIGPGPIPWFIVAELFSQGPRPAAMAVAGFSNWTCNFIVGMGFQYVADAMGPYVFLLFAVLLLGFFIFTFLKVPETRGRTFDQISAAFRRTPSLLEQEVKPSTELEYLGPDEND

SEQ ID NO: 12-19 - skipped (8 entries)

SEQ ID NO: 20 - protein, 11 aa, Mus musculus
VSGWRLFKKIS

SEQ ID NO: 21 - protein, 8 aa, Artificial Sequence
  A synthetic sequence
GSSGGSSG

SEQ ID NO: 22 - DNA, 20 bp, Artificial Sequence
  A synthetic sequence
ctgggtaggcaaggtcctgg